(19) United States
(12) Patent Application Publication
(10) Pub. No.: US 2009/0054828 A1
(43) Pub. Date: Feb. 26, 2009

(54) Systems for transient conduction control

(75) Inventors: Craig Stolen, New Brighton, MN (US); Joseph M. Pastore, Woodbury, MN (US); Robert J. Sweeney, Woodbury, MN (US); Mark Schwartz, White Bear Lake, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., St. Paul, MN (US)

(21) Appl. No.: 11/895,026

(22) Filed: Aug. 22, 2007

(51) Int. Cl. A61N 5/06; A61N 1/30

(52) U.S. Cl. 604/20; 604/522; 607/92

(57) ABSTRACT

The invention provides a system coupled to a heart having a right atrium (RA) and an atrioventricular (AV) node, which includes an implantable gene regulatory signal delivery device configured to deliver a light to a target site in the heart to transiently control an aberrant cardiac electrical conduction, the light having characteristics suitable for regulating a transcription control element; and an implantable medical device communicatively coupled to the implantable gene regulatory signal delivery device, the implantable medical device including: an atrial fibrillation (AF) detector configured to detect AF; and a control circuit configured to initiate an emission of the light from the implantable gene regulatory signal delivery device in response to the detection of AF. Also provided are methods to transiently control aberrant AV conduction or transiently control cardiac arrhythmias, which employ expression cassettes.

### BACKGROUND

Atrial tachyarrhythmias (AT) affect many people and the quality of their lives. For instance, atrial fibrillation (AF) affects an estimated 2.3 million people in the United States. AF is a condition in which control of heart rhythm is taken away from the normal sinus node pacemaker by rapid activity (400-600 pulses per minute in humans versus about 60 beats/minute at rest or 180-200 beats/minute at peak exercise) in different areas within the upper chambers (atria) of the heart. This results in rapid and irregular atrial activity and, instead of contracting, the atria quiver. It is the most common chronic cardiac rhythm disturbance in humans and represents a major clinical problem with serious morbidity and mortality. AF requires a trigger and an atrial substrate to perpetuate AF. Eliminating the trigger or altering the substrate may reduce the incidence of AF. A substrate that perpetuates AF may involve the wavelength (conduction velocity, CV; and effective refractory period, ERP). Altering either CV or ERP may change the substrate necessary to maintain AF. Moreover, short atrial ERPs contribute to the substrate for multiple reentrant wavelets that sustain AF.

Pharmacological and device therapies have not been satisfactory to treat AF, as they have varying degrees of efficacy as well as side effects and complications. Cardiac arrhythmias have been treated traditionally with antiarrhythmic drugs that control the rhythm by altering cardiac electrical properties. However, the available drugs are not specific for atrial electrical activity and can have profound effects on ventricular electrophysiology. For example, K channel blocking drugs that are used to treat AF can mimic potentially lethal congenital disorders of the cardiac repolarization (Such as “torsade-de-pointes”). Moreover, it has become apparent over the last 20 years that the effects of antiarrhythmic drugs on the electrophysiology of the ventricles can themselves paradoxically lead to life-threatening rhythm disorders (proarrhythmia) and increase mortality. Further, drug therapy has only about 60% efficacy. There has been, therefore, a shift towards non-pharmacological therapies for cardiac arrhythmias, including controlled destruction of arrhythmia-generating tissue (“ablation therapy”) and implantable devices that can sense arrhythmias and terminate them with controlled electrical discharges. However, catheter-based therapies are dangerous and highly variable. In contrast to other cardiac arrhythmias, AF continues to be challenge for both pharmacological and non-pharmacological approaches to treatment.

### SUMMARY OF THE INVENTION

The invention provides gene therapy compositions, methods, devices and systems to control the AV node during tachyarrhythmias (e.g., rhythm control during chronic or paroxysmal AF). In one embodiment, a regulatable expression cassette encoding a therapeutic gene product (gene construct) is delivered to cardiac tissue of a mammal, e.g., using an intracoronary approach. In one embodiment, modification of a cardiac electrical substrate includes modification of cardiac sites such as the SA node, fat pads over the AV node, or other sites. The atrial and ventricular rates of the mammal may be monitored with a device, e.g., an internal or external device, and once an aberrant rate is detected, a triggering component which induces transient expression of the expression cassette (e.g., a drug or energy such as light, electromagnetic energy, sound waves, heat and the like) is delivered, e.g., via a device such as an implanted lead, resulting in a therapeutic gene product. The gene product is one which suppresses atrioventricular conduction, thereby slowing the heart rate without producing complete heart block, or which blocks (prevents) atrioventricular conduction, thereby producing a complete but transient heart block. In one embodiment, light induced transient G_{αi2} overexpression suppresses atrioventricular conduction and slows the heart rate without producing complete heart block, or which prevents atrioventricular conduction, thereby producing a complete but transient heart block. Other therapeutic gene products include but are not limited to constitutively active G_{αi2} (Q205L) or GEM a protein that is a calcium channel inhibiting G protein. In one embodiment, device induces expression of inhibitory RNA (e.g., siRNA) specific for an ion channel, e.g., for a Na channel, that may block channel synthesis or of a dominant negative protein which blocks ion channels or gap junction function. In one embodiment, transient expression of a HCN dominant negative subunit suppresses or prevents atrioventricular conduction. Thus, the invention controls AV conduction without permanent ablation of the AV node and subsequent reliance on a back-up VVI pacing device. In one embodiment, the gene therapy compositions may be employed with traditional ICD therapies.

The gene therapy compositions, methods, devices and systems of the invention may be employed to transiently inhibit or treat any arrhythmias including atrial and ventricular arrhythmias. In one embodiment, the gene therapy compositions, methods, devices and systems of the invention may terminate AF by targeting cells that generate early after depolarizations (EADs) or delayed after depolarizations (DADs), or cells that spontaneously trigger activity, or may inhibit or prevent reentry by exciting cells that are part of the reentrant circuit. The gene therapy compositions, methods, devices and systems may also be employed to transiently inhibit or treat disorders associated with aberrant conduction in other electrically active cells or tissues, e.g., nerves or neural tissue, for instance, motor nerves, or in conjunction with ablation, e.g., a Maze-like procedure. In one embodiment, the gene therapy compositions, methods, devices and systems may also be employed to transiently inhibit or treat disorders such as multiple sclerosis, Parkinson's disease, spastic muscles, and the like. The gene therapy compositions, methods and systems may be employed with devices that deliver a regulatory signal such as a drug, including implantable devices, e.g., CRM devices, external devices, and devices having multiple components that are either internal and/or external.

In one embodiment, the mammal is an AF patient being a candidate for AV node ablation and pacemaker implantation. In one embodiment, the mammal is a highly symptomatic paroxsymol AF patient with a pacemaker that utilizes rate smoothing type algorithms. In one embodiment, the mammal is a patient with other ATs (SVT, AFlut and the like). In one embodiment, the mammal is a patient with Wolff-Parkinson-White (WPW) syndrome that is not ablatable. In one embodiment, the mammal is a patient on a AF rhythm control drug that is not tolerating the drug therapy.

Thus, the invention provides compositions, methods, devices, and systems to alter the AV node, thereby inhibiting or treating tachyarrhythmia. The methods may employ vectors, such as viral vectors, to deliver an expression cassette to cells with aberrant conduction properties. The expression cassette has a nucleic acid sequence for a gene product which inhibits conduction, e.g., a nucleic acid sequence corresponding to an inhibitor of beta-adrenergic receptor or an inhibitor of G_{S}, such as RNAi or an antisense oligonucleotide, a Gᵢ protein, a dominant negative inhibitor of an ion channel or gap junction, or a toxin, such as pertussis toxin. In one embodiment, expression of the nucleic acid sequence in the expression cassette in the AV node decreases the amount or activity of beta-adrenergic receptors, G_{S}, an ion channel or gap junction, or increases the amount of Gᵢ, thereby modifying a substrate for arrhythmias and transiently blocking cells from contributing to aberrant conduction. In one embodiment, an afflicted or susceptible AF substrate is modified by delivering a gene encoding a Gᵢ protein, a dominant negative of HCN, or a toxin, or a genetic inhibitor of beta-adrenergic receptor or G_{S}, e.g., beta-adrenergic receptor or G_{S} siRNA or antisense sequences. The delivery of agents that inhibit expression or activity of certain gene products in cardiac cells, inhibit or prevent AT, e.g., by transiently inhibiting or blocking the susceptibility to the triggers for AT.

The vectors with the expression cassette may be delivered systemically, for example, a viral vector may be administered by injection, e.g., to the coronary artery of a mammal. In one embodiment, an expression cassette may be delivered to cardiac tissue via retrograde injection into a coronary vein coeluting blood from the general region where therapy is desired. In one embodiment, an adenoviral vector may be employed. In another embodiment, an adeno-associated viral vector or a lentiviral vector may be employed. The vectors may be delivered locally, e.g., the vectors may be delivered by direct injection into the cardiac muscle where transient blockage of conduction is desired.

In one embodiment, the vector may include a promoter such as one responsive to a stimulus such as electromagnetic energy, light or a drug, which stimulus may be delivered via an interventional cardiology device. The vector may include a tissue-specific transcription control element, for instance, a cardiac-specific or atrial-specific promoter, in addition to a device- or a drug-regulatable transcription control element, and so may be delivered systemically. In one embodiment, genetic inhibitors are expressed by a light inducible promoter. In one embodiment, expression from the light inducible promoter is induced by light from 350 nanometers (nm) to 750 nm, i.e., any one wavelength or a band of wavelengths that include those from 350 up to 750 nm. In one embodiment, expression from the light inducible promoter is induced by red light, e.g., light from 600 to 700 nm. Red light has better penetration than shorter wavelengths of light, and may avoid the damage associated with shorter wavelengths of light and the heat generated by longer wavelengths of light.

In one embodiment, the devices and systems of the invention may include components for automatic AF detection, as well as for activating gene expression, thus allowing for spatial and temporal control of the therapy.

### BRIEF DESCRIPTION OF THE INVENTION

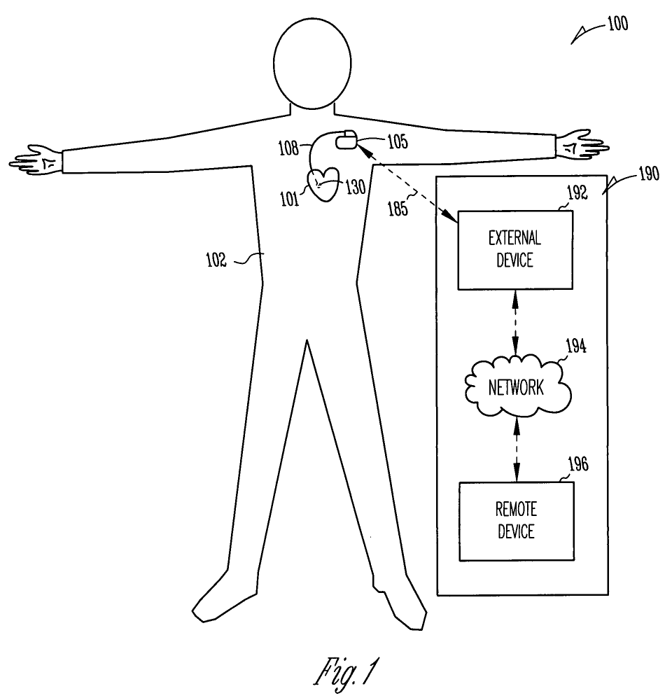

FIG. 1 is an illustration of an embodiment of a gene regulatory system and portions of an environment in which it is used.

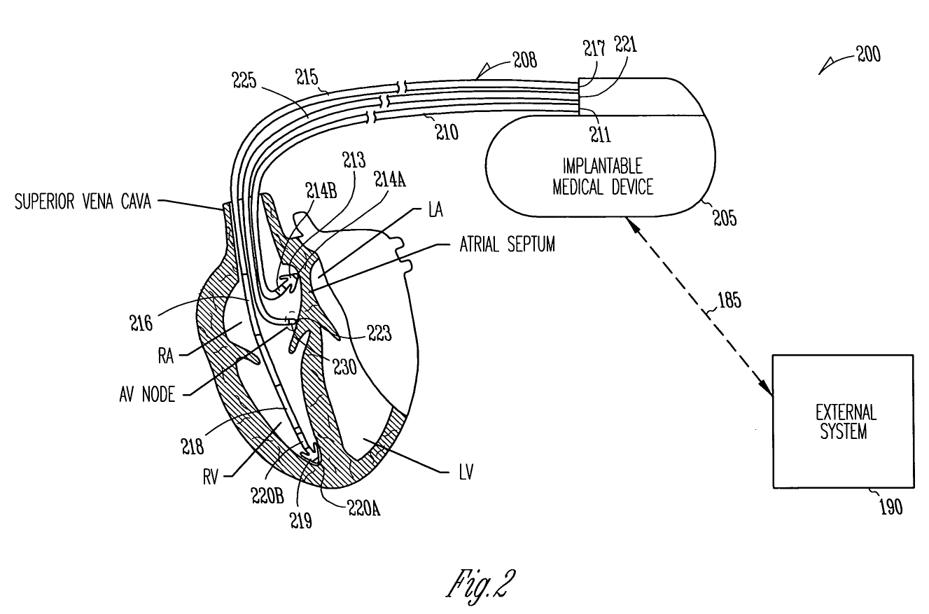

FIG. 2 is an illustration of an embodiment of a cardiac rhythm management (CRM) system including the gene regulatory system and portions of the environment in which the CRM system operates.

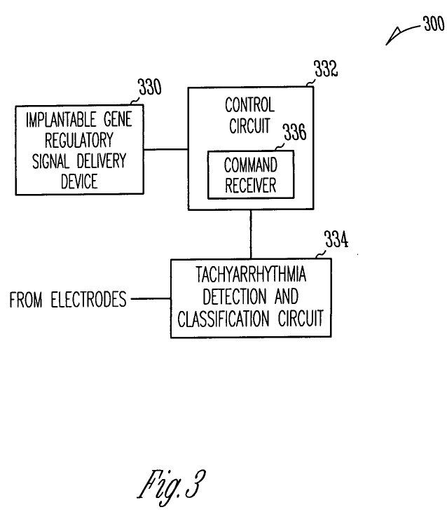

FIG. 3 is a block diagram illustrating an embodiment of the gene regulatory system.

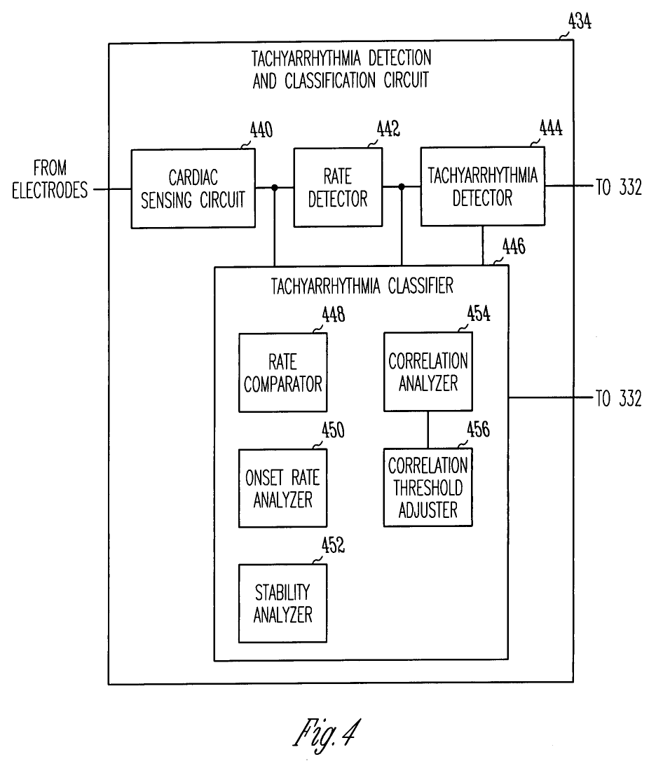

FIG. 4 is a block diagram illustrating an embodiment of a tachyarrhythmia detection and classification circuit of the gene regulatory system.

FIG. 5 is a flow chart illustrating an embodiment of a method for classifying detected tachyarrhythmia.

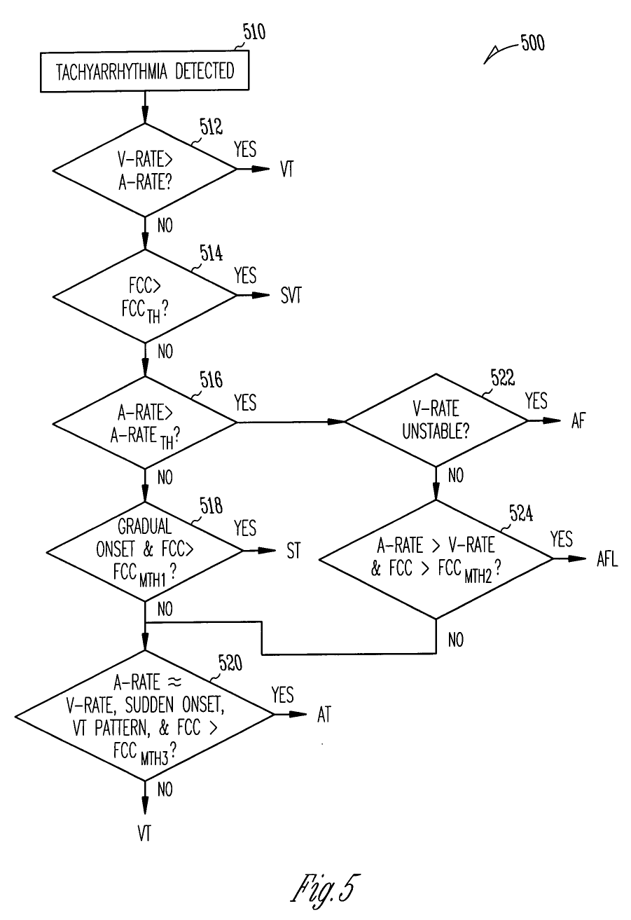

FIG. 6 is a block diagram illustrating an embodiment of a gene regulatory system for gene regulation during AF.

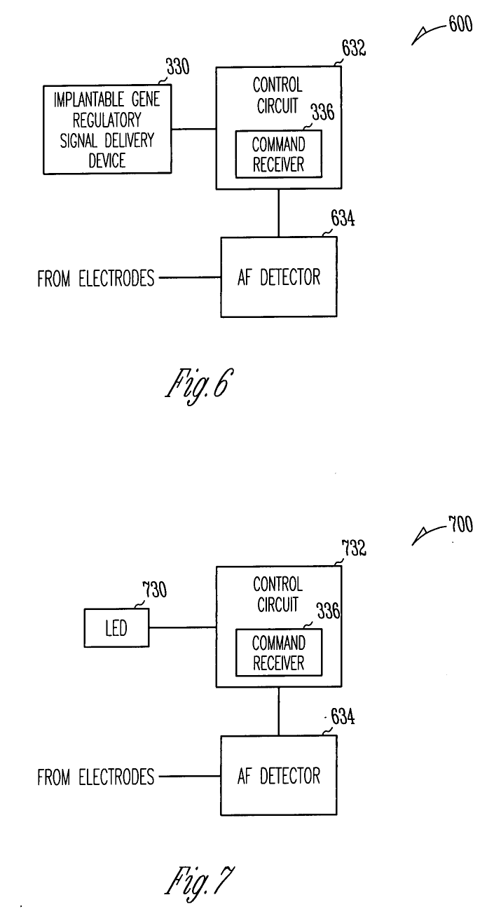

FIG. 7 is a block diagram illustrating a specific embodiment of the gene regulatory system for gene regulation during AF.

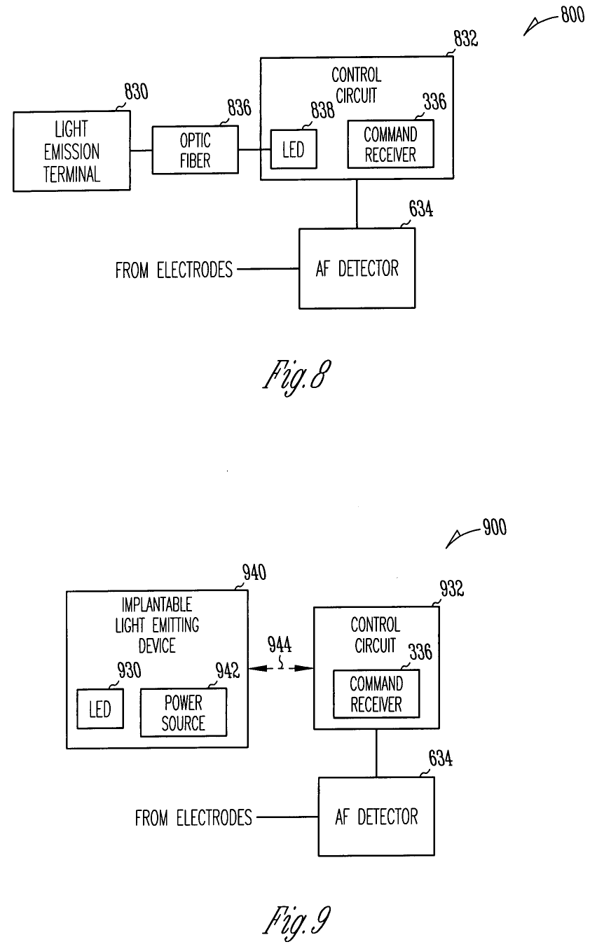

FIG. 8 is a block diagram illustrating another specific embodiment of the gene regulatory system for gene regulation during AF.

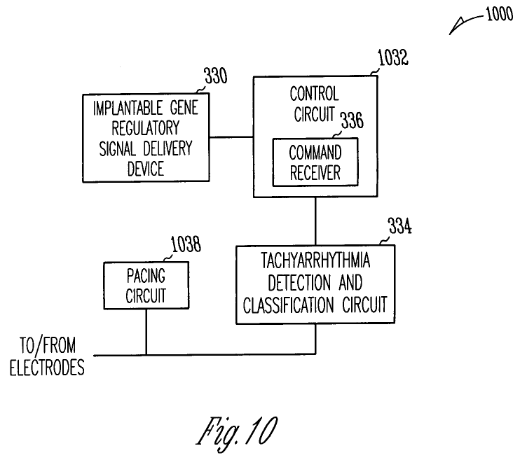

FIG. 9 is a block diagram illustrating another specific embodiment of the gene regulatory system for gene regulation during AF.

FIG. 10 is a block diagram illustrating another specific embodiment of the gene regulatory system for gene regulation during AF.

### DETAILED DESCRIPTION OF THE INVENTION

### DEFINITIONS

By “nucleic acid”, “oligonucleotide”, and “polynucleotide” or grammatical equivalents herein means at least two nucleotides covalently linked together. “Recombinant,” as applied to a polynucleotide, means that the polynucleotide is the product of various combinations of cloning, restriction and/or ligation steps, and other procedures that result in a construct that is distinct from a polynucleotide found in nature. Recombinant as applied to a protein means that the protein is the product of expression of a recombinant polynucleotide.

“In vivo” gene/protein delivery, gene/protein transfer, gene/protein therapy and the like as used herein, are terms referring to the introduction of an exogenous (isolated) polynucleotide or protein directly into the body of an organism, such as a human or non-human mammal, whereby the exogenous polynucleotide or protein is introduced to a cell of such organism in vivo.

The term “corresponds to” is used herein to mean that a polynucleotide or protein sequence is homologous (i.e., may be similar or identical, not strictly evolutionarily related) to all or a portion of a reference polynucleotide or protein sequence. In contradistinction, the term “complementary to” is used herein to mean that the complementary polynucleotide sequence is able to hybridize to the other strand. As outlined below, preferably, the homology between the two sequences is at least 70%, preferably 85%, and more preferably 95%, identical.

The terms “substantially corresponds to” or “substantial identity” or “homologous” as used herein denotes a characteristic of a nucleic acid or protein sequence, wherein a nucleic acid or protein sequence has at least about 70% sequence identity as compared to a reference sequence, typically at least about 85% sequence identity, and preferably at least about 95% sequence identity, as compared to a reference sequence. The reference sequence may be a subset of a larger sequence, such as a portion of a gene or flanking sequence, or portion of protein. However, the reference sequence is at least 20 nucleotides long, typically at least about 30 nucleotides long, and preferably at least about 50 to 100 nucleotides long, or, for peptides or polypeptides, at least 7 amino acids long, typically at least 10 amino acids long, and preferably at least 20 to 30 amino acids long. “Substantially complementary” as used herein refers to a nucleotide sequence that is complementary to a sequence that substantially corresponds to a reference sequence.

“Specific hybridization” is defined herein as the formation of hybrids between a polynucleotide which may include substitutions, deletion, and/or additions as compared to a reference sequence and a selected target nucleic acid sequence, wherein the polynucleotide preferentially hybridizes to a target nucleic acid sequence such that, for example, at least one discrete band can be identified on a Northern or Southern blot of DNA prepared from cells that contain the target nucleic acid sequence. It is evident that optimal hybridization conditions will vary depending upon the sequence composition and length(s) of the polynucleotide(s) and target(s), and the experimental method selected by the practitioner. Various guidelines may be used to select appropriate hybridization conditions.

“Treatment” or “therapy” as used herein refers to administering, to an individual patient, agents that are capable of eliciting a prophylactic, curative or other beneficial effect in the individual.

“Gene therapy” as used herein refers to administering, to an individual patient, vectors comprising a gene encoding a beneficial gene product.

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a sequence of interest for gene therapy. Vectors include, for example, transposons and other site-specific mobile elements, viral vectors, e.g., adenovirus, adeno-associated virus (AAV), poxvirus, papillomavirus, lentivirus, herpesvirus, foamivirus and retrovirus vectors, and including pseudotyped viruses, liposomes and other lipid-containing complexes, and other macromolecular complexes capable of mediating delivery of a polynucleotide to a host cell, e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes. Vectors can also comprise other components or functionalities that further modulate gene delivery and/or gene expression, or that otherwise provide beneficial properties to the cells to which the vectors will be introduced. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described (see, e.g., Curiel et al., Proc. Natl. Acad. Sci. USA, 88:8850 (1991)).

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, iontophoresis, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene may include a gene which is partly or entirely heterologous (i.e., foreign) to the transgenic organism, or may represent a gene homologous to an endogenous gene of the organism.

By “transgenic cell” is meant a cell containing a transgene. For example, a stem cell transformed with a vector containing an expression cassette can be used to produce a population of cells having altered phenotypic characteristics.

The term “wild-type” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

“Vasculature” or “vascular” are terms referring to the system of vessels carrying blood (as well as lymph fluids) throughout the mammalian body.

“Blood vessel” refers to any of the vessels of the mammalian vascular system, including arteries, arterioles, capillaries, venules, veins, sinuses, and vasa vasorum.

“Artery” refers to a blood vessel through which blood passes away from the heart. Coronary arteries supply the tissues of the heart itself, while other arteries supply the remaining organs of the body. The general structure of an artery consists of a lumen surrounded by a multi-layered arterial wall.

The term “transduction” denotes the delivery of a polynucleotide to a recipient cell either in vivo or in vitro, via a viral vector, e.g., via a replication-defective viral vector, such as via a recombinant adenovirus or AAV.

The term “heterologous” as it relates to nucleic acid sequences such as gene sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA, and portions of both double stranded or single stranded sequence. The polynucleotide may be DNA, both genomic and cDNA, RNA or a hybrid, where the polynucleotide contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science, 273:1386 (1996) and Yoon et al., Proc. Natl. Acad. Sci. USA, 93:2071 (1996). It also includes modified polynucleotides such as methylated and/or capped polynucleotides.

A “gene,” “polynucleotide,” “coding region,” or “sequence” which “encodes” a particular gene product, is a nucleic acid molecule which is transcribed and optionally also translated into a gene product, e.g., an antisense sequence or a polypeptide, in vitro or in vivo when placed under the control of appropriate regulatory sequences. The “coding” region may be present in either a cDNA, genomic DNA, RNA form, or a hybrid. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. Thus, a gene includes a polynucleotide which may include a full-length open reading frame which encodes a gene product (sense orientation) or a portion thereof (sense orientation) which encodes a gene product with substantially the same activity as the gene product encoded by the full-length open reading frame, the complement of the polynucleotide, e.g., the complement of the full-length open reading frame (antisense orientation) and optionally linked 5′ and/or 3′ noncoding sequence(s) or a portion thereof, e.g., an oligonucleotide, which is useful to inhibit transcription, stability or translation of a corresponding mRNA. A transcription termination sequence will usually be located 3′ to the gene sequence.

An “oligonucleotide” includes at least 7 nucleotides, preferably 15, and more preferably 20 or more sequential nucleotides, up to 100 nucleotides, either RNA or DNA, which correspond to the complement of the non-coding strand, or of the coding strand, of a selected mRNA, or which hybridize to the mRNA or DNA encoding the mRNA and remain stably bound under moderately stringent or highly stringent conditions, as defined by methods well known to the art, e.g., in Sambrook et al., A Laboratory Manual, Cold Spring Harbor Press (1989).

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence. Thus, a “promoter,” refers to a polynucleotide sequence that controls transcription of a gene or coding sequence to which it is operably linked. A large number of promoters, including constitutive, inducible and repressible promoters, from a variety of different sources, are well known in the art.

By “enhancer element” is meant a nucleic acid sequence that, when positioned proximate to a promoter, confers increased transcription activity relative to the transcription activity resulting from the promoter in the absence of the enhancer domain. Hence, an “enhancer” includes a polynucleotide sequence that enhances transcription of a gene or coding sequence to which it is operably linked. A large number of enhancers, from a variety of different sources are well known in the art. A number of polynucleotides which have promoter sequences (such as the commonly-used CMV promoter) also have enhancer sequences.

By “cardiac-specific enhancer or promoter” is meant an element, which, when operably linked to a promoter or alone, respectively, directs gene expression in a cardiac cell and does not direct gene expression in all tissues or all cell types. Cardiac-specific enhancers or promoters may be naturally occurring or non-naturally occurring. One skilled in the art will recognize that the synthesis of non-naturally occurring enhancers or promoters can be performed using standard oligonucleotide synthesis techniques.

“Operably linked” refers to a juxtaposition, wherein the components so described are in a relationship permitting them to function in their intended manner. By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. Thus, a signal or targeting peptide sequence is operably linked to another protein if the resulting fusion is secreted from a cell as a result of the presence of a secretory signal peptide or into an organelle as a result of the presence of an organelle targeting peptide.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide or virus refers to a nucleic acid sequence, peptide, polypeptide or virus that is identified and separated from at least one contaminant nucleic acid, polypeptide, virus or other biological component with which it is ordinarily associated in its natural source. Isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may be single-stranded), but may contain both the sense and anti-sense strands (i.e., the molecule may be double-stranded).

The term “peptide”, “polypeptide” and protein” are used interchangeably herein unless otherwise distinguished to refer to polymers of amino acids of any length. These terms also include proteins that are post-translationally modified through reactions that include glycosylation, acetylation and phosphorylation.

“Gene regulation” or “Gene regulatory therapy” as used herein includes delivery of one or more gene regulatory signals to regulate gene expression in a gene therapy vector. The gene regulatory signals include signals that trigger a transcriptional control element, e.g., a promoter.

### General Overview

In a normal, healthy heart, cardiac contraction is initiated by the spontaneous excitation of the sinoatrial (“SA”) node, located at the junction of the right atrium and superior vena cava (“SVC”). The electrical impulse generated by the SA node travels to the atrioventricular (“AV”) node where it is transmitted to the bundle of His and Purkinje network, which branches in many directions to facilitate simultaneous contraction of the left and right ventricles.

A depolarizing wave front gives rise to contractions among the myocytes of the atria and ventricles. During normal contraction, the left and right atria simultaneously contract followed by the simultaneous contraction of the left and right ventricles to eject oxygenated blood from the left ventricle to the aorta. In certain disease states, the heart's ability to properly pace the atria and ventricles is compromised.

Intracellular electrical conduction is governed by either influx of positive ions causing activation of a cell or electrical communication between cells via gap junctions. This document describes, among other things, compositions, methods, devices and systems for the control of AF gene therapy, in which a therapeutic gene construct with a regulatable transcription control element such as a promoter (e.g., drug or light) is inserted into the cell, and the activation of the transcription control element results in inhibitory RNA that blocks ion (e.g., Na) channel synthesis or alternatively, a dominant negative protein that blocks ion (e.g., Na) channel opening or gap junction function which inhibits or prevents conduction. In one embodiment, a mammal having or at risk of having AF is subjected to gene therapy which is intended to transiently inhibit or treat AF. The gene therapy vector encodes at least one gene product that is operably linked to at least one regulatable transcription control element, forming an expression cassette. In one embodiment, the gene therapy vector includes at least one transgene that encodes a gene product corresponding to a dominant negative gap junction protein or an ion channel protein, a genetic inhibitor of beta-adrenergic receptor or G_{S} including antisense sequences, for instance, an antisense oligonucleotide or siRNA, or Gᵢ or toxin proteins, e.g., pertussis toxin. The expression of the gene product is under the control of a regulatable transcription control element, such as an inducible promoter or an enhancer. For example, the gene product is under control of a light responsive promoter. In one embodiment, the vector encoding the gene product is systemically applied, while light is locally applied, to control conduction in cells or tissue with aberrant conduction. In another embodiment, the vector is locally administered to cells or tissue with aberrant conduction properties, e.g., administered to the pulmonary vein, and light may be broadly applied. In one embodiment, the expression of the gene is also tissue-specific, e.g., cardiac cell-specific, due to a tissue-specific promoter and/or enhancer. For instance, the enhancer may be a muscle creatine kinase (mck) enhancer, or the promoter may be an alpha-myosin heavy chain (MyHC) or beta-MyHC promoter (see Palermo et al., Circ. Res., 78, 504 (1996)).

In comparison to current, less than optimal therapies for AF, the present invention provides for genetic intervention. Genetic intervention is advantageous because relevant genes may be specifically targeted to the atria, gene expression thereof may be regulatable with specific transcriptional control elements, it is less traumatic and has reduced surgical complications.

In one embodiment, the invention provides methods, devices and systems for suppressing gap junction activity because gap junctions are responsible for the intercellular transfer of electrical current. Connexin proteins are a family of homologous proteins found in connexins of gap junctions as homo- or heterohexameric arrays. Connexins are pore-like complex protein structures forming channels (gap junctions) between cells. Each cell contributes one hemi-channel to form a connexin. Connexin proteins are the major gap junction protein involved in the electrical coupling of myocardial cells. Gap junctions regulate intercellular passage of molecules, including inorganic ions and second messengers, thus achieving electrical coupling of cells. Connexin subunit isoforms can vary in size between about 25 kDa and 60 kDa and generally having four putative transmembrane spanning regions. Different connexins are specific for various parts of the heart.

Connexin proteins found in the cardiovascular system include connexin 37 (“Cx37”), connexin 40 (“Cx40”), connexin 43 (“Cx43”), and connexin 45 (“Cx45”) See, Van Veen et al., Cardiovascular Research, 51:217 (2001).; Severs et al., Microscopy Research and Technique, 52:301 (2001); Kwong et al., Circulation Research, 82:604 (1998)). The primary connexin isoforms found in the heart are connexin 40, 43 and 45.

Other parts of the heart also utilize the connexin proteins as a gap junction protein involved in the electrical coupling of cells. In the crista terminalis, a part of the normal right atrium of the heart, the predominant connexin isoform in the atrium is connexin 43. Here, the conduction velocities may be as high as 1.2 m/sec and connexin 43 is believed to account for the increased conduction velocities. Moreover, in the Purkinje fiber system, conduction velocities are even higher (2.4 m/s), and the predominant connexin isoform found is connexin 40.

Currents across gap junctions are also regulated and gated by a variety of factors, such as pH, voltage, intracellular calcium and phosphorylation. Indeed, even the intercellular coupling of other ion channels, such as sodium channels, effect conduction velocities. However, the role of connexins is key to conductivity of electrical pulses in the heart and the amount of connexin produced is central to regulation of electrical stimulation of the myocytes.

The invention provides methods, devices and systems which express dominant negative proteins that form gap junctions or interfere with gap junction activity, or encode products that block ion channels, such as Na channels. In one embodiment, the expression cassette encodes a dominant negative connexin, e.g., one having Q49K, L90V, R202H, or V216L, G21R, G138R, or G60S of Cx43, a frame shift at 260Cx43, a stop codon at R33 of Cx43, a deletion of residues 130-137 in Cx43. Examples of Cx43 sequences include those of Genbank Accession Nos. XP027460, XP027459, XP004121, P17302, AAD37802, A35853, NP000156, AF151980, M65188, and AAA52131).

In one embodiment, the nucleotide sequences encode a dominant negative hyperpolarization-activated cation channel protein (HCN) or a portion thereof. Four isoforms of the HCN family, HCN1, HCN2, HCN3, and HCN4 have been identified. The HCN4 isoform may be the predominant subunit encoding for the cardiac funny current channel in the SA node. In one embodiment, the expression cassette encodes a dominant negative HCN having G365A:Y366A:G367A.

A variety of dominant negative proteins can be prepared for use in the methods of the invention. For example, ion channel proteins are recognized as one protein family for which dominant negative proteins can be readily made, e.g., by removing selected transmembrane domains. In most cases, the function of the ion channel binding complex is substantially reduced or eliminated by interaction of a dominant negative ion channel protein. For example, a DNA encoding a protein comprising one or more transmembrane domains is modified so that at least 1 and preferably 2, 3, 4, 5, 6 or more of the transmembrane domains are eliminated. The resulting modified protein may form a binding complex with at least one other protein and inhibit normal function of the binding complex, e.g., as assayed by standard ligand binding assays or electrophysiological assays. A dominant negative protein may exhibit at least 10 percent or greater inhibition of the activity of the binding complex; more preferably at least 20 percent or greater; and still more preferably at least about 30, 40, 50, 60, 70, 80, or 100 percent or greater inhibition of the binding complex activity relative to the full-length protein.

In another embodiment, a cDNA encoding a desired protein for use in the present methods can be modified so that at least one amino acid of the protein is deleted or substituted by a conservative or non-conservative amino acid. For example, a tyrosine amino acid substituted with a phenylalanine would be an example of a conservative amino acid substitution, whereas an arginine replaced with an alanine would represent a non-conservative amino acid substitution.

In one embodiment, the vector encodes a genetic inhibitor of beta-adrenergic receptor or G_{S}. Thus, the invention provides systems for localized beta-blockade, including a device and gene therapy construct to control the AV node during tachyarrhythmias. The β₁-adrenoceptors and a majority of other cardiovascular receptors identified to date belong to the guanine nucleotide binding (G) protein-coupled receptor families that mediate signaling by coupling primarily to three G proteins, the stimulatory (G_{S}), inhibitory (Gᵢ), and G_{q/11} proteins to stimulate the adenylate cyclases and phospholipases, activating a small but diverse subset of effectors and ion channels.

β₁ subtype is the most prominent adrenergic receptor in the heart as β₁-adrenergic receptor signaling to initiate arrhythmia. All the β-adrenergic receptors are G protein-coupled receptors (GPCRs). G proteins are three subunits, α, β, and γ. The α-subunit binds to guanine nucleotides (GTP) and catalyzes enzymatic conversion to guanosine diphosphate (GDP). In their inactive state, G proteins are found as an αβγ trimer bound to GDP. When an agonist binds to the receptor, the trimer is recruited to the intracellular loop region, resulting in the dissociation of GDP and the subsequent binding of GTP. The G protein trimer then breaks up its active α-GTP and βγ-subunit forms which diffuse into the cytosol to activate (or inactivate) enzymes or channel proteins. β-subtypes differ in terms the second messengers that transmit the adrenergic signal. The classic β-adrenergic receptor signaling pathway involves coupling to G_{αs}, which in turns activates adenylyl cyclase.

Phase 0 of the cardiac action potential consists of rapid depolarization caused opening of cardiac Na⁺ channels (I_{Na}) followed by activation outward K⁺ currents during phase 1, e.g., the transient outward current (Iₜₒ), and the ultra-rapid delayed rectifier (I_{Kur}). This is followed by activation of phase 2 currents. During this phase, inward currents such as the L-type calcium current (I_{CaL}), and the sodium current (I_{Na,L}) balance outward currents such as I_{Kur}, and the rapidly activating and slowing activating components of the delayed rectifier current, I_{Kr} and I_{Ks}. Ca²⁺ influx during the plateau phase is essential electromechanical coupling. Phase 3 is the final rapid repolarization and dominated by the outward K⁺ currents I_{Kr} and I_{Ks}. Maintenance of the resting membrane during phase 4 is controlled by the inward rectifier current (I_{K1}). In regions with pacemaker activity, the hyperpolarization-activated current I_{f} is able to depolarize cells during phase 4.

Cardiac arrhythmias are believed to arise by four primary mechanisms: early after depolarizations (EADs), delayed after depolarizations (DADs), enhanced automaticity, and reentry. Triggered activity occurring before full repolarization of the AP is termed an EAD. DADs can occur in the ventricles, Purkinje fibers, and the atria and are typically caused by Ca²⁺ overload. Under normal conditions, the SAN controls cardiac rate because of its faster intrinsic firing rate compared to other regions; however, regions like the AVN and the His-Purkinje system are capable of depolarizing spontaneously and displaying automaticity.

Reentry is a disorder of impulse conduction that is believed to cause many important clinical tachyarrhythmias. Once normal tissue is excited, the Na⁺ channels become inactivated and another AP cannot be initiated until they recover from inactivation—a period of time termed the refractory (RP). There are three main requirements for initiation of reentry: (1) two distinct pathways for AP propagation joined proximally and distally; (2) different RPs in the two pathways; and (3) development of unidirectional block, generally by premature activations exposing the RP differences.

The methods of the subject invention utilize oligonucleotides and RNA nucleic acid pharmaceutical compositions that interfere with the expression or activity of beta-adrenergic receptor or G_{S}. The oligonucleotides are designed to elicit strong and specific suppression of beta-adrenergic receptor or G_{S} gene expression in mammalian cells.

RNA interference is a post-transcriptional process triggered by the introduction of double-stranded RNA which leads to gene silencing in a sequence-specific manner. RNA interference reportedly occurs naturally in organisms as diverse as nematodes, trypanosomes, plants and fungi. It is believed to protect organisms from viruses, modulate transposon activity and eliminates aberrant transcription products.

In one embodiment, a siRNA approach is employed. The siRNA interacts with helicase and nuclease to form a complex termed “RNA-induced silencing complex” (RISC). RISC then unwinds the double-stranded siRNA. Antisense then binds to target RNA, which is then cleaved by RISC. The target RNA is further degraded by cellular nucleases. This process is known as RNA interference or RNAi.

The effectiveness of siRNAs of the most potent siRNAs result in greater than 90% reduction in target RNA and protein levels. See e.g., Caplen et al., Proc. Natl. Acad. Sci. USA, 98:9746 (2001); Elbashir et al., Nature, 411:494 (2001); Holen et al., Nucleic Acids Research, 30:1757 (2002). Certain proven siRNAs that have been shown to be very effective contain 21 bp dsRNAs with 2 short 3′ overhangs. The effectiveness of the siRNA depends on structure and position. See Brown et al., TechNotes, 9:3 (2002); Holen et al., Nucleic Acids Research, 30:1757 (2002); Jarvis et al., TechNotes, 8:3 (2001).

For instance, target sequences of 21 nucleotides that are located within a region of the coding sequence that is within 50-100 nucleotides of start codon and within 50-100 nucleotides from the termination codon are selected. The presence of AA at the start sequence allows for the use of dTdT at the 3′ end of the antisense sequence. The sense strand can be synthesized with dTdT at the 3′ end, because only the antisense strand is involved in target recognition. Moreover, the use of dTdT makes the siRNA duplex more resistant to exonucleic activity. The G-C content of a particular sequence may also be used for selecting target sequences. The content may be less than 50%, e.g., in the 40% range, although successful gene silencing has been reported with siRNA having between 50 and 60% G-C content. Sequences with repeats of three or more G's or C's are generally avoided, as their presence may initiate molecular secondary structures preventing effective siRNA silencing hybridization. Stretches of A's and T's may also be avoided. Target sequences that have more than 15 contiguous nucleotide sequence identity to other known genes are avoided.

In one embodiment, the construct encodes a protein product (e.g., inhibitory G proteins (Gᵢ)). In one embodiment, the construct encodes an inhibitory protein such as a pertussis toxin.

In one embodiment, a therapeutic gene is delivered to the AV node, e.g., via catheterization of the right coronary artery, and VEGF, nitroglycerin, and an adenovirus with the therapeutic gene. The gene therapy construct includes a device or drug regulated promoter and an open reading frame for a conduction inhibiting product. In one embodiment, concurrent with or after gene therapy, a device which is capable regulating expression of the gene(s) in the gene therapy vector is provided to the mammal. Arrhythmias may be sensed with a device, e.g., a device which senses atrial rate and uses algorithms to control AV node conduction accordingly. In response to detection of an arrhythmia, e.g., a change in a physiological parameter indicative of fibrillation, the device emits a signal which activates a regulatable transcription control element in the gene therapy vector. Such signals include, but are not limited to, electromagnetic field, light, and/or a drug. In one embodiment, the regulatable transcription control element is a light inducible element from a human gene, a plant gene, a fungi, an invertebrate, or is synthetic. In one embodiment, a light activation system is derived from Neurospora, e.g., from white collar complex.

In another embodiment, the regulatable transcription control element is regulated by a drug, e.g., one delivered systemically or by an implantable device. In one embodiment, the gene construct contains inducible genetic elements (e.g., a tetracycline (tet) responsive promoter) that allow its expression to be controlled by an orally administered drug, e.g., tet or an analog or derivative thereof. The construct is delivered to the AV node via a catheter, and the patient is monitored with a device (e.g., holter monitor, Polar chest band, or implanted device). After sensing an arrhythmic event the device may signal the patient to take the drug (e.g., tet), the device may be coupled with a latitude repeater to allow a physician to monitor and adjust the therapy. Because the drug is degraded via normal drug pharmacokinetics, the expression may be short lived and the effect can be transient, e.g., dependent on RNA, as well as non-cytotoxic (reversible). In one embodiment, the construct may be delivered to a chamber of the heart following a “Genetic tattoo maze procedure,” and the encoded antisense siRNA decreases expression of proteins associated with impulse conduction.

In one embodiment, a signal is delivered to the tissue to turn on gene expression (e.g., via an implanted lead with an LED shining on the AV node). In one embodiment, the signal is delivered via a lead directed to the tissue where expression (transient conduction modification/ablation) is desired. In one embodiment, a single light application may discriminate between atrial and ventricular origin. In one embodiment, the signal is light comprised of one or more spectral frequencies. The invention includes the use of multiple compounds sensitive to different light frequencies, and the use of multiple light sources to control intensity or to act on different sites. In one embodiment, after expression from the gene therapy vector is induced and a desirable change in the physiological parameter detected, the signal is discontinued. In another embodiment, the signal is emitted for a predetermined time period. In another embodiment, light of particular wavelength(s) are used to induce gene expression while light of different wavelength(s) are emitted to turn off gene expression. Thus, gene expression may be turned on and off or titrated by controlling signals emitted by the device.

Thus, the use of gene therapy compositions, devices and systems of the invention reduce irregular ventricular intervals that may occur during atrial arrhythmias, and reduce symptoms seen in chronic or paroxysmal AF patients. The invention provides rate control without permanent ablation of AV node or the need for chronic ventricular pacing.

To evaluate the efficacy of gene therapy, immediately before gene transfer and one week afterward, a steerable quadripolar electrophysiological (EP) catheter may be placed into the high right atrium, a non-steerable EP catheter may be placed into the right ventricle, and a non-steerable EP catheter may be placed into the His-bundle position. Baseline intracardiac electrograms are obtained, and electrocardiographic intervals are recorded. Following standard techniques, the AVNERP is measured by programmed stimulation of the right atrium.

After baseline measurements are obtained, atrial fibrillation is induced by burst atrial pacing and decrementing over a short period.

To evaluate the efficacy of gene therapy, a mammalian heart (in vivo or ex vivo) is contacted with an expression cassette of the invention. After exposure to a drug- or device-based regulatory signal modulation (increase or decrease) of at least one electrical property in the heart is detected, e.g., at least one of heart rate, conduction velocity, refractory period, firing rate and/or pulse rate, relative to a baseline value.

### Delivery of Conduction Altering Gene Constructs

In one embodiment, the expression cassette is delivered to the cardiac tissue via direct injection into a coronary artery supplying a region of tissue where the expression (transient modification of conduction) is desired. In one embodiment, the expression cassette is delivered to the cardiac tissue via direct injection into the cardiac muscle in the region of tissue where the expression (transient modification of conduction) is desired. In one embodiment, the expression cassette is delivered to the cardiac tissue via injection into the pericardial space. In one embodiment, the expression cassette is delivered to the cardiac tissue via retrograde injection into a coronary vein collecting blood from the general region of tissue where the expression is desired. In one embodiment, the expression cassette is delivered systemically by intravenous injection. In another embodiment, the expression cassette is delivered via a catheter following a “genetic tattoo maze procedure,” e.g., the vector is applied in a specific pattern, such as a pattern following the burn lines generated in ablation procedures, for instance, around the pulmonary vein. After sensing an arrhythmic event the device or drug can stimulate gene expression. Production of antisense or siRNA reduces expression of proteins associated with impulse conduction. Because RNA expression is short lived and is device or drug regulated, the effect can be transient, non-cytotoxic, and painless.

### Gene Therapy Vectors

Gene therapy vectors include, for example, viral vectors, liposomes and other lipid-containing complexes, and other macromolecular complexes capable of mediating delivery of a gene to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Gene therapy vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene therapy vectors are described below. Gene therapy vectors may be administered via any route including, but not limited to, intramuscular, buccal, rectal, intravenous or intracoronary administration, and transfer to cells may be enhanced using electroporation and/or iontophoresis.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing cardiac specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. Moreover, they appear promising for sustained cardiac gene transfer (Hoshijima et al,. Nat. Med., 8:864 (2002); Lynch et al., Circ. Res., 80:197 (1997)).

### Herpesvirus/Amiplicon

Herpes simplex virus 1 (HSV-1) has a number of important characteristics that make it an important gene delivery vector in vivo. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has obviously strong tropism towards nerve cells. It has a very large genome size and can accommodate very large transgenes (>35 kb). Herpesvirus vectors are particularly useful for delivery of large genes, e.g., genes encoding ryanodine receptors and titin.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Synthetic Oligonucleotides

Antisense oligonucleotides are short (approximately 10 to 30 nucleotides in length), chemically synthesized DNA molecules that are designed to be complementary to the coding sequence of an RNA of interest. These agents may enter cells by diffusion or liposome-mediated transfer and possess relatively high transduction efficiency. These agents are useful to reduce or ablate the expression of a targeted gene while unmodified oligonucleotides have a short half-life in vivo, modified bases, sugars or phosphate groups can increase the half-life of oligonucleotide. For unmodified nucleotides, the efficacy of using such sequences is increased by linking the antisense segment with a specific promoter of interest, e.g., in an adenoviral construct. In one embodiment, electroporation and/or liposomes are employed to deliver plasmid vectors. Synthetic oligonucleotides may be delivered to cells as part of a macromolecular complex, e.g., a liposome, and delivery may be enhanced using techniques such as electroporation.

### Regulatable Transcription Control Elements

The device of the invention may deliver one or more signals including, but not limited to, light of a particular wavelength or a range of wavelengths, light of a particular energy, acoustic energy, an electric field, a chemical, electromagnetic energy, thermal energy or other forms of temperature or matter, which signal is recognized by a regulatable transcription control element in a gene therapy vector. In one embodiment, the signal is a chemical. In one embodiment, the signal is thermal energy. In one embodiment, the signal is electrical energy. In one embodiment, the signal is acoustic energy. In one embodiment, the signal is ultrasound. In one embodiment, the signal is RF.

A variety of strategies have been devised to control in vivo expression of transferred genes and thus alter the pharmacokinetics of in vivo gene transfer vectors in the context of regulatable or inducible promoters. Many of these regulatable promoters use exogenously administered agents to control transgene expression and some use the physiologic milieu to control gene expression. Examples of the exogenous control promoters include the tetracycline-responsive promoter, a chimeric transactivator consisting of the DNA and tetracycline-binding domains from the bacterial tet repressor fused to the transactivation domain of herpes simplex virion protein 16 (Ho et al., Brain Res. Mol. Brain Res., 41:200 (1996)); a chimeric promoter with multiple cyclic adenosine monophosphate response elements superimposed on a minimal fragment of the 5′-flanking region of the cystic fibrosis transmembrane conductance regulator gene (Suzuki et al., 7:1883 (1996)); the EGR1 radiation-inducible promoter (Hallahan et al., Nat. Med., 1:786 (1995)); and the chimeric GRE promoter (Lee et al., J. Thoracic Cardio. Surg., 118:26 (1996)), with 5 GREs from the rat tyrosine aminotransferase gene in tandem with the insertion of Ad2 major late promoter TATA box-initiation site (Narumi et al., Blood, 92:812 (1998)). Examples of the physiologic control of promoters include a chimera of the thymidine kinase promoter and the thyroid hormone and retinoic acid-responsive element responsive to both exogenous and endogenous tri-iodothyroniine (Hayashi et al., J. Biol. Chem., 269:23872 (1994)); complement factor 3 and serum amyloid A3 promoters responsive to inflammatory stimuli; the grp78 and BiP stress-inducible promoter, a glucose-regulated protein that is inducible through glucose deprivation, chronic anoxia, and acidic pH (Gazit et al., Cancer Res., 55:1660 (1995)); and hypoxia-inducible factor 1 and a heterodimeric basic helix-loop-helix protein that activates transcription of the human erythropoietin gene in hypoxic cells, which has been shown to act as a regulatable promoter in the context of gene therapy in vivo (Forsythe et al., Mol. Cell Biol., 16:4604 (1996)).

Regulatable transcription elements useful in gene therapy vectors and methods of the invention include, but are not limited to, a truncated ligand binding domain of a progesterin receptor (controlled by antiprogestin), a tet promoter (controlled by tet and dox) (Dhawan et al., Somat. Cell. Mol. Genet., 21, 233 (1995); Gossen et al., Science, 268:1766 (1995); Gossen et al., Science, 89:5547 (1992); Shockett et al., Proc. Natl. Acad. Sci. USA, 92, 6522 (1995)), hypoxia-inducible nuclear factors (Semenza et al., Proc. Natl. Acad. Sci. USA, 88, 5680 (1991); Semenza et al., J. Biol. Chem., 269, 23757)), steroid-inducible elements and promoters, such as the glucocorticoid response element (GRE) (Mader and White, Proc. Natl. Acad. Sci. USA, 90, 5603 (1993)), and the fusion consensus element for RU486 induction (Wang et al., Proc. Natl. Acad. Sci. USA, 91:818 (1994)), those sensitive to electromagnetic fields, e.g., those present in metallothionein I or II, c-myc, and HSP70 promoters (Lin et al., J. Cell. Biochem., 81:143 (2001); Lin et al., J. Cell. Biochem., 54:281 (1994); U.S. published application 20020099026)), and electric pulses (Rubenstrunk et al., J. Gene Med., 5:773 (2003)), as well as a yeast GAL4/TATA promoter, auxin inducible element, an ecdysone responsive element (No et al., Proc. Natl. Acad. Sci. USA, 93:3346 (1996)), an element inducible by rapamycin (FK506) or an analog thereof (Rivera et al., Nat. Med., 2:1028 (1996); Ye et al., Science, 283:88 (1999); Rivera et al., Proc. Natl. Acad. Sci. USA, 96:8657 (1999)), a tat responsive element, a metal, e.g., zinc, inducible element, a radiation inducible element, e.g., ionizing radiation has been used as the inducer of the promoter of the early growth response gene (Erg-1) (Hallahan et al., Nat. Med., 1:786 (1995)), an element which binds nuclear receptor PPARγ (peroxisome proliferators activated receptors), which is composed of a minimal promoter fused to PPRE (PPAR responsive elements, see WO 00/78986), a cytochrome P450/A1 promoter, a MDR-1 promoter, a promoter induced by specific cytokines (Varley et al., Nat. Biotech., 15:1002 (1997)), a light inducible element (Shimizu-Sato et al., Nat. Biotech., 20:1041 (2002)), a lacZ promoter, and a yeast Leu3 promoter.

In one embodiment, the regulatable transcription control element is regulated by light. Light regulated genes include but are not limited to phytolyases, phytochromes, white collar complex (WCC), cryptochromes, phototropins, mimecan, chalcone synthases (CHS), encephalopsin, photoactive yellow protein, and dark stipe.

CPD photolyases repair cyclobutane pyrimidine dimers (CPDs) induced in DNA. Photolyases are found in many organisms including but not limited to E. coli, A. nidulans, P. tridactylus, D. melanogaster, O. latipes, C. auratus, M. domestica, T. harzianum, and S. cerevisiae. Photolyases contain one flavin adenine dinucleotide (FAD) and either a methenyltetrahydrofolate (MTHF, type-1 photolyases) or an 8-hydroxy-5-deazariboflavin (type-2 photolyases). The photolyase binds the DNA dimer and the coenzyme receives blue light (350 to 500 nm). The induction of the photolyase gene is very rapid (about 15 to 30 minutes) and significant. The system may be altered by substituting flavins responsive to other wavelengths of light.

Phytochromes are protein complexes composed of bilin chromophores. The bilin chromophore (light-sensing structure) is a linear tetrapyrrole (four 5-carbon rings covalently bonded) which is synthesized from heme by several enzymes. The apophytochrome protein spontaneously binds the chromophore in the cell cytoplasm to form the phytochrome complex. This is a covalent association via a thioether linkage. The phytochrome has 1-3 PAS domains involved in protein-protein interaction and nuclear localization, a GAF domain, and a PHY domain at the N-terminus and a histidine kinase-related domain (HKRD) at the C-terminus (Rockwell et al., 2006). When the phytochrome complex is exposed to seconds to minutes of red light of about 660 nm, the inactive form of the complex (P_{R}) undergoes isomerization to the active (P_{FR}) form. This exposes the nuclear localization signal in the PAS domain, allowing for nuclear localization where the N-terminal domains interact with transcription factors. When the P_{FR} phytochrome complex is exposed to seconds to minutes of far red light of about 750 nm, it converts back to the P_{R} form and leaves the nucleus, stopping gene regulation. Alternately, if the P_{FR} phytochrome complex is left with no light stimulation for several hours it will revert to the P_{R} form. Phytochrome complexes can be found in all flowering plants and cryptophytes, cyanobacteria, nonoxygenic bacteria, and fungi. A tyrosine-to-histidine mutation of the phytochrome causes it to give off an intense red fluorescence when excited by light.

For instance, U.S. Pat. No. 6,887,688 discloses a cell with hemeoxygenase and a ferredoxin-dependent bilin reductase (such as PcyA or HY2) to produce the bilin component of the phytochrome complex, a gene for the C-terminal PAS domain of the phytochrome (which functions as an nuclear localization signal (NLS)) genetically combined with an N-terminal transcription factor of choice, and a target gene with a promoter which corresponds to the transcription factor.

U.S. patent application No. 2003/0082809A1 discloses a cell with a phytochrome genetically engineered with a DNA-binding domain (DBD, constitutively expressed, a chromophore (expressed or added exogenously), a phytochrome interacting factor (PIF) genetically engineered with an activating domain (AD, constitutively expressed), and a target gene with a promoter which corresponds to the activating domain. The phytochrome-DBD binds to the target gene and in the presence of red light it interacts with the PIF-AD to initiate transcription of the target gene. It ceases to interact with the PIF-AD in the presence of far red light, stopping transcription of the target gene.

The white collar-1 (WC-1) and white collar-2 (WC-2) proteins are transcriptional regulators. They bind promoters through GATA-type zinc-finger DNA binding-domains, and they complex with one another through PAS domains. One PAS domain on WC-1 is a member of the light, oxygen, or voltage (LOV) class, and is responsible for binding to flavin adenine dinucleotide (FAD). FAD serves as the blue light sensor for the white collar complex with peak responsiveness at 370 and 450 nm.

U.S. Pat. No. 6,733,996 describes a method for using the WCC to regulate gene expression. This invention involves a cell containing FAD (all cells have FAD) engineered with the WC-1 and WC-2 genes genetically linked to be expressed as a fusion protein in which the zinc-finger DBD of WC-1 is replaced with a different transactivator, and a target gene linked to a promoter element which corresponds to the transactivator.

Cryptochromes serve as blue light photoreceptors in both prokaryotes and eukaryotes. The cryptochrome (cry 1 and 2) have C-terminal extensions not found in photolyases. These C-terminal domains mediate a constitutive light response. It is hypothesized that these domains are in an inactive state in the dark and blue light relieves the repression through an intra- or intermolecular redox reaction with the flavin chromophore. Cry 1 binds to FAD, which may serve as its chromophore. Cry 2 is strongly downregulated by blue light. Cry 1 and 2 are known to be involved in light sensing in the retina.

Phototrophins are membrane-bound kinases in plants which contain LOV (light, oxygen, voltage) PAS domains and bind FMN (flavin mononucleotide) to sense blue light. Light appears to cause a conformational change in phototrophins, exposing the PAS domains and activating kinase function, and allowing regulating of phototrophism in plants.

Promoters or other transcription control elements regulated by light useful in the compositions, methods, and systems of the invention include but are not limited to those disclosed in U.S. Pat. No. 6,858,429 (red or far-red light; 600 nm to 750 nm); U.S. Pat. No. 6,733,996 (430 nm to 480 nm); U.S. Pat. No. 6,887,688; a photolyase system which is chromophore-based system for DNA damage repair with a FAD cofactor, that is expressed rapidly after blue light exposure (350 nm to 500 nm); a phytochrome system which is a chromophore-based system that has a protein complex which interconverts in response to red and far red light (about 660 nm to about 750 nm); white collar complex, in which WC-1 and WC-2 bind FAD and regulate gene expression (450 nm to 470 nm); a cryptochrome system found in circadian clock mechanism and plant functions (broad UV-A band and blue light); a phototropin (nph1) system, where light activates protein kinase function; a human mimecan promoter, where encoded protein is induced about 24 hours after UV exposure; a CHS promoter, which is induced by UV light; an encephalopsin system; a photoactive yellow protein (maximum at 446 nm); and a dark-stipel (dstl) system (UV and blue light). Thus, the methods and systems of the invention may include the use of other expression cassettes to express heterologous gene products that confer light responsiveness.

In one embodiment, a gene expression system that is to be used for the delivery of device-regulated light-inducible gene therapy is rapidly and significantly inducible by light, tightly regulated, has low/no basal expression, and/or shuts off in the absence of light rapidly. In one embodiment, the light regulated transcription control element binds Pfr when exposed to red light. Thus, cardiac cells may include expression cassettes for phytochrome apoprotein, e.g., PcyA or Hy2, and optionally other proteins found in the complex that binds the light regulated transcription control element, WC1 and WC2 or other light responsive protein that binds to a promoter, or a fusion protein having a transcription factor binding protein fused to a light sensitive protein.

In one embodiment, a device that emits light from 350 to 500 nm, or any one or band of wavelengths from 350 to 500 nm, may be employed with a photolyase responsive promoter. In one embodiment, a device that emits light from 630 to 690 nm, or any one or band of wavelengths from 630 to 690 nm, may be employed with a photochrome responsive promoter. In one embodiment, a device that emits light from 430 to 490 nm, or any one or band of wavelengths from 430 to 490 nm, may be employed with a WCC responsive promoter. In one embodiment, a device that emits a broad UV band or blue light may be employed with a cryptochrome responsive promoter. In one embodiment, a device that emits UV light may be employed with a mimican, CHS or dstl responsive promoter. In one embodiment, a device that emits light from 420 to 450 nm, or any one or band of wavelengths from 420 to 450 nm, may be employed with a photoactive yellow protein responsive promoter.

In some embodiments, cell- or tissue-specific control elements, such as muscle-specific and inducible promoters, enhancers and the like, will be of particular use, e.g., in conjunction with regulatable transcriptional control elements. Such control elements include, but are not limited to, those derived from the actin and myosin gene families, such as from the myoD gene family (Weintraub et al., Science, 251, 761 (1991)); the myocyte-specific enhancer binding factor MEF-2 (Cserjesi and Olson, Mol. Cell Biol., 11, 4854 (1991)); control elements derived from the human skeletal actin gene (Muscat et al., Mol. Cell Bio., 7, 4089 (1987)) and the cardiac actin gene; muscle creatine kinase sequence elements (Johnson et al., Mol. Cell Biol., 9, 3393 (1989)) and the murine creatine kinase enhancer (mCK) element; control elements derived from the skeletal fast-twitch troponin C gene, the slow-twitch cardiac troponin C gene and the slow-twitch troponin I genes.

Cardiac cell restricted promoters include but are not limited to promoters from the following genes: a α-myosin heavy chain gene, e.g., a ventricular α-myosin heavy chain gene, β-myosin heavy chain gene, e.g., a ventricular β-myosin heavy chain gene, myosin light chain 2v gene, e.g., a ventricular myosin light chain 2 gene, myosin light chain 2a gene, e.g., a ventricular myosin light chain 2 gene, cardiomyocyte-restricted cardiac ankyrin repeat protein (CARP) gene, cardiac α-actin gene, cardiac m2 muscarinic acetylcholine gene, ANP gene, BNP gene, cardiac troponin C gene, cardiac troponin I gene, cardiac troponin T gene, cardiac sarcoplasmic reticulum Ca-ATPase gene, skeletal α-actin gene, as well as an artificial cardiac cell-specific promoter.

Further, chamber-specific promoters or enhancers may also be employed, e.g., for atrial-specific expression, the quail slow myosin chain type 3 (MyHC3) or ANP promoter, or the cGATA-6 enhancer, may be employed. For ventricle-specific expression, the iroquois homeobox gene may be employed. Examples of ventricular myocyte-specific promoters include a ventricular myosin light chain 2 promoter and a ventricular myosin heavy chain promoter.

In other embodiments, disease-specific control elements may be employed. Thus, control elements from genes associated with a particular disease, including but not limited to any of the genes disclosed herein may be employed in vectors of the invention.

Nevertheless, other promoters and/or enhancers which are not specific for cardiac cells or muscle cells, e.g., RSV promoter, may be employed in the expression cassettes and methods of the invention. Other sources for promoters and/or enhancers are promoters and enhancers from the Csx/NKX 2.5 gene, titin gene, α-actinin gene, myomesin gene, M protein gene, cardiac troponin T gene, RyR2 gene, Cx40 gene, and Cx43 gene, as well as genes which bind Mef2, dHAND, GATA, CarG, E-box, Csx/NKX 2.5, or TGF-beta, or a combination thereof.

The response of the regulatable transcriptional control element to one or more intermittent signals, a prolonged signal or different levels of a signal, may be tested in vitro or in vivo. The vector may include the regulatable transcriptional control element linked to a marker gene, i.e., one which is readily detectable or capable of detection such as green fluorescent protein (GFP). For example, a vector having a promoter which is sensitive to electrical pulses, a MT-I or MT-II promoter (Rubenstruck et al., J. Gene Med., 5:773 (2003)), is linked to an open reading frame for a marker gene. The resulting expression cassette, e.g., one which is introduced to an adenovirus vector or to a plasmid vector, is employed to infect or transfect murine cells, e.g., murine cardiac cells, or heart sections. An electrode system designed for use in a small flask is used to deliver electrical pulses. Then fluorescence in the cells or a lysate thereof is detected, and/or or vector specific RNA is measured, for instance, using RT-PCR, and optionally compared to data from control cells. Similarly, a vector having a promoter which is sensitive to electrical pulses is linked to an open reading frame for a therapeutic gene, e.g., Serca2, introduced to cells, e.g., cardiac cells such as those with decreased levels of the gene product encoded by the therapeutic gene, and the phenotype of the recombinant cells compared to control cells. Vectors may also be introduced to a non-human large animal model, e.g., pigs, to determine the level and spatial expression of the exogenously introduced gene in response to signals, e.g., electrical pulses, from an implantable device in that animal.

### Vector Delivery

Several techniques have been developed for cardiac gene delivery, including pericardial infusion, endomyocarial injection, intracoronary injection, coronary venous retroperfusion, and aortic root injection (Isner, Nature, 415:234 (2002)). The different techniques achieve variable response in homogeneity of gene delivery, resulting in focal gene expression within the heart (Hajjar et al., Circ. Res., 86:616 (2000). For this reason, techniques that achieve diffuse uptake would seem to be superior. Two such methods utilize the heart's arterial and venous circulation to accomplish disseminated viral transfection. Arterial injection, performed directly through a percutaneous approach or indirectly by an infusion into the cross-clamped aorta, has shown promise in animal models of heart failure and is appealing in that it can be performed either at the time of cardiac surgery or as percutaneous intervention (Hajjar et al., PNAS USA, 95:5251 (1998)). Similarly, retroperfusion through the coronary sinus appears to produce a more global gene expression in comparison with techniques of localized or focal injection (Boeckstegers et al., Circ., 100:1 (1999)).

The expression cassette may be administered intravenously, transvenously, intramyocardially or by any other convenient route, and delivered by a needle, catheter, e.g., a catheter which includes an injection needle or infusion port, or other suitable device.

### Direct Myocardial Injection

Direct myocardial injection of plasmid DNA as well as virus vectors, e.g., adenoviral vectors, and cells including recombinant cells has been documented in a number of in vivo studies. This technique when employed with plasmid DNA or adenoviral vectors has been shown to result in effective transduction of cardiac myocytes. Thus, direct injection may be employed as an adjunct therapy in patients undergoing open-heart surgery or as a stand-alone procedure via a modified thorascope through a small incision. Virus, e.g., pseudotyped, or DNA- or virus-liposome complexes may be delivered intramyocardially.

### Catheter-Based Delivery

Intracoronary delivery of genetic material can result in transduction of approximately 30% of the myocytes predominantly in the distribution of the coronary artery. Parameters influencing the delivery of vectors via intracoronary perfusion and enhancing the proportion of myocardium transduced include a high coronary flow rate, longer exposure time, vector concentration, and temperature. Gene delivery to a substantially greater percent of the myocardium may be enhanced by administering the gene in a low-calcium, high-serotonin mixture (Donahue et al., Nat. Med., 6:1395 (2000)). The potential use of this approach for gene therapy for heart failure may be increased by the use of specific proteins that enhance myocardial uptake of vectors (e.g., cardiac troponin T).

Improved methods of catheter-based gene delivery have been able to achieve almost complete transfection of the myocardium in vivo. Hajjar et al. (Proc. Natl. Acad. Sci. USA, 95:5251 (1998)) used a technique combining surgical catheter insertion through the left ventricular apex and across the aortic valve with perfusion of the gene of interest during cross-clamping of the aorta and pulmonary artery. This technique resulted in almost complete transduction of the heart and could serve as a protocol for the delivery of adjunctive gene therapy during open-heart surgery when the aorta can be cross-clamped.

### Pericardial Delivery

Gene delivery to the ventricular myocardium by injection of genetic material into the pericardium has shown efficient gene delivery to the epicardial layers of the myocardium. However, hyaluronidase and collagenase may enhance transduction without any detrimental effects on ventricular function. Recombinant cells may also be delivered pericardially.

### Intravenous Delivery

Intravenous gene delivery may be efficacious for myocardial gene delivery. However, to improve targeted delivery and transduction efficiency of intravenously administered vectors, targeted vectors may be employed. In one embodiment, intravenous administration of DNA-liposome or antibody-DNA complexes may be employed.

### Lead-Based Delivery

Gene delivery can be performed by incorporating a gene delivery device or lumen into a lead such as a pacing lead, defibrillation lead, or pacing-defibrillation lead. An endocardial lead including a gene delivery device or lumen allows gene delivery to the endocardial layers of the myocardium. An epicardial lead including a gene delivery device or lumen allows gene delivery to the endocardial layers of the myocardium. A transvenous lead including a gene delivery device or lumen may also allow intravenous gene delivery. Lead-based delivery is particularly advantageous when the lead is used to deliver electrical and gene therapies to the same region.

Generally any route of administration may be employed, including oral, mucosal, intramuscular, buccal and rectal administration. For certain vectors, certain route of administration may be preferred. For instance, viruses, e.g., pseudotyped virus, and DNA- or virus-liposome, e.g., HVJ-liposome, may be administered by coronary infusion, while HVJ-liposome complexes may be delivered pericardially.

### Dosages and Dosage Forms

The amount of gene therapy vector(s) administered and device based signal emitted to achieve a particular outcome will vary depending on various factors including, but not limited to, the gene and promoter chosen, the condition, patient specific parameters, e.g., height, weight and age, and whether prevention or treatment is to be achieved. The gene therapy vector/device system of the invention is amenable to chronic use for prophylactic purposes.

Vectors of the invention may conveniently be provided in the form of formulations suitable for administration, e.g., into the blood stream (e.g., in an intracoronary artery). A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, preferably about 10⁹ viral particles, and more preferably about 10¹¹ viral particles. The number of viral particles may, but preferably does not exceed 10¹⁴. As noted, the exact dose to be administered is determined by the attending clinician, but is preferably in 1 ml phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

In one embodiment, in the case of heart disease, administration may be by intracoronary injection to one or both coronary arteries (or to one or more saphenous vein or internal mammary artery grafts or other conduits) using an appropriate coronary catheter. A variety of catheters and delivery routes can be used to achieve intracoronary delivery, as is known in the art. For example, a variety of general purpose catheters, as well as modified catheters, suitable for use in the present invention are available from commercial suppliers. Also, where delivery to the myocardium is achieved by injection directly into a coronary artery, a number of approaches can be used to introduce a catheter into the coronary artery, as is known in the art. By way of illustration, a catheter can be conveniently introduced into a femoral artery and threaded retrograde through the iliac artery and abdominal aorta and into a coronary artery. Alternatively, a catheter can be first introduced into a brachial or carotid artery and threaded retrograde to a coronary artery. Detailed descriptions of these and other techniques can be found in the art (see, e.g., above, including: Topol, (ed.), The Textbook of Interventional Cardiology, 4th Ed. (Elsevier 2002); Rutherford, Vascular Surgery, 5th Ed. (W. B. Saunders Co. 2000); Wyngaarden et al. (eds.), The Cecil Textbook of Medicine, 22nd Ed. (W. B. Saunders, 2001); and Sabiston, The Textbook of Surgery, 16th Ed. (Elsevier 2000)).

By way of illustration, liposomes and other lipid-containing gene delivery complexes can be used to deliver one or more transgenes. The principles of the preparation and use of such complexes for gene delivery have been described in the art (see, e.g., Ledley, Human Gene Therapy, 6:1129 (1995); Miller et al., FASEB Journal, 9:190 (1995); Chonn et al., Curr. Opin. Biotech., 6:698 (1995); Schofield et al., British Med. Bull., 51:56 (1995); Brigham et al., J. Liposome Res., 3:31 (1993)).

Administration of the gene therapy vector in accordance with the present invention may be continuous or intermittent, depending, for example, upon the recipient's physiological condition, whether the purpose of the administration is therapeutic or prophylactic, and other factors known to skilled practitioners. The administration of the gene therapy vector may be essentially continuous over a preselected period of time or may be in a series of spaced doses. Both local and systemic administration is contemplated.

One or more suitable unit dosage forms comprising the gene therapy vector, which may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

Pharmaceutical formulations containing the gene therapy vector can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions for convenient oral administration or as solutions appropriate for parenteral administration, for instance by intramuscular, subcutaneous or intravenous routes.

The pharmaceutical formulations of the vectors can also take the form of an aqueous or anhydrous solution or dispersion, or alternatively the form of an emulsion or suspension.

Thus, the vector may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the vectors may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The vector may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antimicrobial agents or preservatives.

### Device

FIGS. 1-9 illustrate a gene regulatory system providing for transient control of conduction in portions of a cardiac electric conduction system. In one embodiment, the gene regulatory system is part of an implantable cardiac rhythm management (CRM) system and delivers a gene regulatory signal to the AV node to control the AV conduction, such as delaying or blocking the AV conduction during an AF episode.

As used in this document, the relationship between a heart rate and a cardiac cycle length (also known as cardiac interval) is the relationship between a frequency and its corresponding period. If a heart rate is given in beats per minute (bpm), its corresponding cardiac cycle length in milliseconds is calculated by dividing 60,000 by the heart rate (where 60,000 is the number of milliseconds in a minute). Any process, such as a comparison, using a heart rate is to be modified accordingly when a cardiac cycle length is used instead. For example, if a tachyarrhythmia is detected when the ventricular rate exceeds a tachyarrhythmia threshold rate, an equivalent process is to detect the tachyarrhythmia when the ventricular cycle length (also known as ventricular interval) falls below a tachyarrhythmia threshold interval. The appended claims should be construed to cover such variations.

In this document, a “fast beat” refers to a heart beat having a heart rate that falls into a tachyarrhythmia detection zone, which is typically defined by at least one tachyarrhythmia detection threshold, and a “slow beat” refers to a heart beat having a heart rate that is below the tachyarrhythmia detection zone. In other words, a “fast beat” is a heart beat having a tachyarrhythmic heart rate, and a “slow beat” is a heart beat having a heart rate that is not tachyarrhythmic.

FIG. 1 is an illustration of an embodiment of a gene regulatory system 100 and portions of an environment in which it is used. System 100 includes an implantable medical device 105, an implantable gene regulatory signal delivery device 130, a lead system 108, an external system 190, and a telemetry link 185. Telemetry link 185 provides for communication between implantable medical device 105 and external system 190.

As shown in FIG. 1, implantable medical device 105 is implanted in a patient's body 102. Implantable medical device 105 includes a gene regulatory controller that controls a gene therapy. Implantable gene regulatory signal delivery device 130 delivers the gene regulatory signal to portions of the electrical conduction system of heart 101. Lead system 108 provides for access to one or more locations to which the one or more gene regulatory signals are delivered. In one embodiment, lead system 108 also includes one or more leads providing for electrical connections between implantable medical device 105 and heart 101 to allow delivery of electrical therapies in addition to the gene regulatory signal. In various embodiments, in addition to the gene regulatory controller, implantable medical device 105 also includes a pacemaker, a cardioverter/defibrillator, a cardiac resynchronization therapy (CRT) device, a cardiac remodeling control therapy (RCT) device, a neurostimulator, a drug delivery device or a drug delivery controller, a cell therapy device, and/or any other sensing or therapeutic component. Lead system 108 further includes leads for sensing physiological signals and delivering pacing pulses, cardioversion/defibrillation shocks, neurostimulation pulses, and/or pharmaceutical or other substances.

In the illustrated embodiment, implantable gene regulatory signal delivery device 130 is incorporated into lead system 108 for localized delivery of the gene regulatory signal. In another embodiment, implantable gene regulatory signal delivery device 130 is placed in a location relatively remote from the treated site and delivers the gene regulatory signal to reach the treated site via the circulatory system or through tissue. In a specific embodiment, implantable gene regulatory signal delivery device 130 is incorporated into implantable medical device 105.

External system 190 includes an external device 192, a telecommunication network 194, and a remote device 196. External device 192 is within the vicinity of implantable medical device 105 and communicates with implantable medical device 105 bi-directionally via telemetry link 185. Remote device 196 is in a remote location and communicates with external device 192 bi-directionally via network 194, thus allowing a user to monitor and treat a patient from a distant location.

System 100 allows the delivery of the one or more gene regulatory signals to be triggered by any one of implantable medical device 105, external device 192, and remote device 196. In one embodiment, implantable medical device 105 triggers the delivery of the gene regulatory signal upon detecting a predetermined signal or condition, such as an arrhythmia episode. In another embodiment, external device 192 or remote device 194 triggers the delivery of the gene regulatory signal upon detecting an abnormal condition from a signal transmitted from implantable medical device 105. In a specific embodiment, external system 190 includes a processor running a therapy decision algorithm to determine whether and when to trigger the delivery of the gene regulatory signal. In another specific embodiment, external system 190 includes a user interface to present signals acquired by implantable medical device 105 and/or the detected abnormal condition to a user and receives commands from the user for triggering the delivery of the gene regulatory signal. In another specific embodiment, the user interface includes a user input incorporated into external device 192 to receive commands from the user and/or the patient treated with system 100. For example, the patient may be instructed to enter a command for the gene regulatory signal when he senses certain symptoms, and another person near the patient may do the same upon observing the symptoms.

It is to be understood that an implantable gene regulatory signal delivery device and an implantable medical device are discussed to illustrate, but not to restrict, the present subject matter. Though discussed specifically as part of a CRM system, the gene regulatory system and method discussed in this document is generally usable for all in vivo gene therapies delivered by implantable or external devices.

FIG. 2 is an illustration a CRM system 200 and portions of an environment in which system 200 operates. CRM system 200 includes an implantable medical device 205, an implantable gene regulatory signal delivery device 230, a lead system 208, and external system 190. Lead system 208 includes implantable leads 210, 215, and 225.

Implantable medical device 205 represents an embodiment of implantable medical device 105 and includes a hermetically sealed can housing an electronic circuit that senses physiological signals and delivers therapies. In one embodiment, implantable medical device 205 delivers electrical therapies and gene regulatory signal initiated therapies. The hermetically sealed can also finction as an electrode for sensing and/or electrical pulse delivery purposes. In one embodiment, implantable medical device 205 includes an arrhythmia detection circuit that detects tachyarrhythmias and determines whether a gene regulatory signal is to be delivered from implantable medical device 205. For example, if AF is detected, implantable medical device 205 causes implantable gene regulatory signal delivery device 230 to deliver one or more gene regulatory signals to the AV node to delay AV conduction. If VF is detected, implantable medical device 205 delivers a defibrillation therapy. In various embodiments, in addition to controlling or delivering the gene therapy, implantable medical device 205 is capable of delivering pacing, cardioversion/defibrillation, neurostimulation, drug, and other biologic therapies.

Lead 210 is an implantable intracardiac right atrial (RA) pacing lead that includes an elongate lead body having a proximal end 211 and a distal end 213. Proximal end 211 is coupled to a connector for connecting to implantable medical device 205. Distal end 213 is configured for placement in the RA in or near the atrial septum. Lead 210 includes an RA tip electrode 214A, and an RA ring electrode 214B. RA electrodes 214A and 214B are incorporated into the lead body at distal end 213 for placement in or near the atrial septum, and are each electrically coupled to implantable medical device 205 through a conductor extending within the lead body. RA tip electrode 214A, RA ring electrode 214B, and/or the can of implantable medical device 205 allow for sensing an RA electrogram indicative of RA depolarizations and delivering RA pacing pulses.

Lead 215 is an implantable intracardiac right ventricular (RV) pacing-defibrillation lead that includes an elongate lead body having a proximal end 217 and a distal end 219. Proximal end 217 is coupled to a connector for connecting to implantable medical device 205. Distal end 219 is configured for placement in the RV. Lead 215 includes a proximal defibrillation electrode 216, a distal defibrillation electrode 218, an RV tip electrode 220A, and an RV ring electrode 220B. Defibrillation electrode 216 is incorporated into the lead body in a location suitable for supraventricular placement in the RA and/or the superior vena cava. Defibrillation electrode 218 is incorporated into the lead body near distal end 219 for placement in the RV. RV electrodes 220A and 220B are incorporated into the lead body at distal end 219. Electrodes 216, 218, 220A, and 220B are each electrically coupled to implantable medical device 205 through a conductor extending within the lead body. Proximal defibrillation electrode 216, distal defibrillation electrode 218, and/or the can of implantable medical device 205 allow for delivery of cardioversion/defibrillation pulses to the heart. RV tip electrode 220A, RV ring electrode 220B, and/or the can of implantable medical device 205 allow for sensing an RV electrogram indicative of RV depolarizations and delivering RV pacing pulses.

Lead 225 is an implantable intracardiac gene regulatory lead that includes an elongate lead body having a proximal end 221 and a distal end 223. Proximal end 221 is coupled to a connector for connecting to implantable medical device 205. Distal end 223 is configured for placement in the RA over the AV node. Implantable gene regulatory signal delivery device 230 is incorporated into distal end 223.

Leads 210, 215, and 225 are shown in FIG. 2 for illustrative purposes only. Other lead configurations also allow proper delivery of the electrical and gene therapies. In one embodiment, implantable gene regulatory signal delivery device 230 is incorporated into a lead for pacing and/or cardioversion/defibrillation.

Implantable gene regulatory signal delivery device 230 represents an embodiment of implantable gene regulatory signal delivery device 130 and delivers one or more gene regulatory signals to the AV node. In one embodiment, implantable gene regulatory signal delivery device 230 also generates the one or more gene regulatory signals. In another embodiment, implantable medical device 205 generates the one or more gene regulatory signals, which are transmitted through lead 225 to implantable gene regulatory signal delivery device 230 for delivery to the AV node.

CRM system 200 may be implemented using a combination of hardware and software. In various embodiments, electrical elements of CRM system 200 may each be implemented using an application-specific circuit constructed to perform one or more particular functions or a general-purpose circuit programmed to perform such function(s). Such a general-purpose circuit includes, but is not limited to, a microprocessor or portions thereof, a microcontroller or portions thereof, and a programmable logic circuit or portions thereof. For example, a “comparator” includes, among other things, an electronic circuit comparator constructed to perform the only function of comparing two or more signals or a portion of a general-purpose circuit driven by a code instructing that portion of the general-purpose circuit to perform the comparing.

FIG. 3 is a block diagram illustrating an embodiment of a gene regulatory system 300 being part of system 100 or 200. System 300 includes an implantable gene regulatory signal delivery device 330, a control circuit 332, and a tachyarrhythmia detection and classification circuit 334.

Implantable gene regulatory signal delivery device 330 represents a specific embodiment of gene regulatory signal delivery device 130 or 230 and delivers one or more gene regulatory signals having characteristics suitable for providing transient control of the AV conduction by the gene regulation discussed above. In one embodiment, implantable gene regulatory signal delivery device 330 is incorporated into the distal end of a lead such as illustrated in FIG. 2. In one embodiment, implantable gene regulatory signal delivery device 330 generates the one or more gene regulatory signals and delivers the one or more gene regulatory signals to the AV node. In another embodiment, control circuit 332 generates the one or more gene regulatory signals and transmits the one or more gene regulatory signals to gene regulatory signal delivery device 330 through the lead. Gene regulatory signal delivery device 330 delivers the one or more gene regulatory signals to the AV node.

In one embodiment, implantable gene regulatory signal delivery device 330 includes an electric field generator that generates and emits an electric field. The electric field has predetermined frequency and strength selected for regulating gene expression to temporary control the AV conduction. In one specific embodiment, an electric field generator includes electrodes to which a voltage is applied. The intensity of the electric field is controlled by controlling the voltage across the electrodes.

In one embodiment, implantable gene regulatory signal delivery device 330 includes an electromagnetic field generator that generates and emits an electromagnetic field. The electromagnetic field has predetermined frequency and strength selected for regulating gene expression to temporary control the AV conduction. In one specific embodiment, the electromagnetic field generator includes an inductive coil. The intensity of the electromagnetic field is controlled by controlling the voltage across the coil and/or the current flowing through it. In one specific embodiment, the electromagnetic field has a frequency of about 1 Hz to 1 KHz. In another specific embodiment, the electromagnetic field is a direct-current (dc) electromagnetic field.

In one embodiment, implantable gene regulatory signal delivery device 330 includes an optical emitter that emits light. The light has predetermined wavelength or band of wavelengths and intensity selected for regulating gene expression to temporarily control the AV conduction. In one embodiment, the optical emitter includes a light-emitting diode (LED), which is further discussed with reference to FIG. 7. In one embodiment, the optical emitter includes a light-emitting xenon flash tube. In one embodiment, the optical emitter includes a laser.

In one embodiment, implantable gene regulatory signal delivery device 330 includes a speaker that emits a sound. The sound has a predetermined frequency and intensity selected for regulating gene expression to temporarily control the AV conduction.

In one embodiment, implantable gene regulatory signal delivery device 330 includes a drug delivery device which emits one or more chemical agents. The one or more chemical agents have properties known to regulate expression to temporarily control the AV conduction. Examples of the one or more chemical agents include chemicals which induce expression from a particular promoter, including tetracycline, rapamycin, auxins, metals and ecdysone.

In one embodiment, implantable gene regulatory signal delivery device 330 includes a thermal radiator that emits a thermal energy. The thermal energy changes the tissue temperature to a point or range suitable for regulating gene expression to temporarily control the AV conduction. In one specific embodiment, the thermal radiator includes a resistive element that is heated as electrical current flows through it or as a voltage is applied across it. The tissue temperature is controlled by controlling the amplitude of the electrical current or voltage.

In one embodiment, implantable gene regulatory signal delivery device 330 includes a heat sink that absorbs thermal energy. The thermal energy absorption changes the tissue temperature to a point or range suitable for regulating gene expression to temporarily control the AV conduction. In one specific embodiment, the heat sink includes peltier cooler that absorb heat as electrical current flows through it. The tissue temperature is controlled by controlling the polarity of the current and the amplitude of the electrical current or voltage.

Control circuit 332 controls the delivery of the one or more gene regulatory signals from implantable gene regulatory signal delivery device 330. In one embodiment, control circuit 332 initiates, adjusts, and/or stops the delivery of the one or more gene regulatory signals in response to events detected and classified by tachyarrhythmia detection and classification circuit 334. In one embodiment, as illustrated in FIG. 3, control circuit 332 includes a command receiver 336 to receive an external command for initiating, adjusting, and/or stopping the delivery of the one or more gene regulatory signals. In various embodiments, the external command is issued by a physician or other caregiver, such as through a user interface of external system 190, or by the patient, such as by a handheld therapy control device. In one embodiment, events detection and classification by tachyarrhythmia detection and classification circuit 334 are communicated to external system 190 to inform the physician or other caregiver, who in turn may enter the external command. Instead of, or in addition to, initiating the delivery of the one or more gene regulatory signals, the physician or other caregiver may also administrate one or more other therapies, such as a drug therapy. In another embodiment, the patient is informed of a need for therapy by external system 190, or feels such a need. The patient may use the handheld therapy control device, such as a magnet, to initiate the delivery of the one or more gene regulatory signals.

Tachyarrhythmia detection and classification circuit 334 detects and classifies tachyarrhythmia episode using at least one or more cardiac signals sensed using electrodes such as those illustrated in FIG. 2. In one embodiment, in addition to one or more cardiac signals, tachyarrhythmia detection and classification circuit 334 uses one or more other physiological signals, such as one or more signals indicative of hemodynamic performance, to detect and classify tachyarrhythmia episode.

FIG. 4 is a block diagram illustrating an embodiment of a tachyarrhythmia detection and classification circuit 434. Tachyarrhythmia detection and classification circuit 434 is a specific embodiment of tachyarrhythmia detection and classification circuit 334 and includes a cardiac sensing circuit 440, a rate detector 442, a tachyarrhythmia detector 444, and a tachyarrhythmia classifier 446.

Cardiac sensing circuit 440 senses one or more cardiac signals, such as one or more electrograms, using electrodes such as those illustrated in FIG. 2. In one embodiment, cardiac sensing circuit 440 is electrically coupled to heart 101 through leads 205 and 210 to sense an atrial electrogram and a ventricular electrogram from the heart. The atrial electrogram includes atrial events, also known as P waves, each indicative of an atrial depolarization. The ventricular electrogram includes ventricular events, also known as R waves, each indicative of a ventricular depolarization.

Rate detector 442 detects one or more heart rates from one or more cardiac signals sensed by cardiac sensing circuit 440. In one embodiment, rate detector 442 detects an atrial rate from the atrial electrogram and a ventricular rate from the ventricular electrogram. The atrial rate is the frequency of the atrial events. The ventricular rate is the frequency of the ventricular events. In one embodiment, the atrial and ventricular rates are each expressed in beats per minute (bpm), i.e., number of detected atrial or ventricular depolarizations per minute.

Tachyarrhythmia detector 444 detects a tachyarrhythmia episode. In one embodiment, a tachyarrhythmia is detected when the ventricular rate exceeds a predetermined tachyarrhythmia threshold rate. In one embodiment, tachyarrhythmia detector 444 detects tachyarrhythmia by determining whether the ventricular rate is within one of a plurality of tachyarrhythmia rate zones each including a predetermined threshold rate. In a specific embodiment, the plurality of tachyarrhythmia rate zones includes a ventricular fibrillation (VF) rate zone with a VF threshold rate programmable between 130 and 250 bpm, a fast ventricular tachycardia (VT) rate zone with a fast VT threshold rate programmable between 110 and 210 bpm, and a slow VT rate zone with a slow VT threshold rate programmable between 90 and 200 bpm. In another embodiment, the tachyarrhythmia is detected using a “zoneless tachyarrhythmia detection” method, as discussed in U.S. patent application Ser. No. 11/301,716, “ZONELESS TACHYARRHYTHMIA DETECTION WITH REAL-TIME RHYTHM MONITORING”, filed on Dec. 13, 2005, assigned to Cardiac Pacemakers, Inc., which is incorporated herein by reference in its entirety.

Tachyarrhythmia classifier 446 classifies each tachyarrhythmia detected by tachyarrhythmia detector 444. Examples of classification of tachyarrhythmia made by tachyarrhythmia classifier 446 include ventricular fibrillation (VF), ventricular tachycardia (VT), supraventricular tachyarrhythmia (SVT), atrial fibrillation (AF), atrial flutter (AFL), sinus tachycardia (ST), and atrial tachycardia (AT). In one embodiment, a detected tachyarrhythmia is classified as VF when the ventricular rate falls within the VF rate zone, without further analysis by tachyarrhythmia classifier 446. In the illustrated embodiment, tachyarrhythmia classifier 446 includes a rate comparator 448, an onset rate analyzer 450, a stability analyzer 452, a correlation analyzer 454, and a correlation threshold adjuster 456. Rate comparator 448 compares the atrial rate and the ventricular rate to determine whether the atrial rate exceeds, equals, or is lower than the ventricular rate by a predetermined margin. Onset rate analyzer 450 produces an onset rate of the detected tachyarrhythmia and determines whether the detected tachyarrhythmia has a gradual onset or a sudden onset by comparing the onset rate to one or more threshold onset rates. The onset rate is a rate of transition of the ventricular rate from a normal sinus rate to a tachyarrhythmic rate when the detected tachyarrhythmia begins. A gradual onset typically indicates a physiological tachyarrhythmia, such as an ST caused by exercise. A sudden onset typically indicates a pathological tachyarrhythmia. Stability analyzer 452 produces a stability parameter indicative of a degree of ventricular rate variability and determines whether the ventricular rate is stable by comparing the stability parameter to a stability threshold. In one embodiment, the stability parameter is produced as an average variance of a series of ventricular intervals. Correlation analyzer 454 analyzes a correlation between a tachyarrhythmic waveform and a template waveform and produces a correlation coefficient representative of that correlation. The tachyarrhythmic waveform includes a segment of a cardiac signal sensed during the detected tachyarrhythmia. The template waveform is recorded during a known cardiac rhythm such as the normal sinus rhythm (NSR). One example for producing such a correlation coefficient, referred to as a feature correlation coefficient (FCC), is discussed in U.S. Pat. No. 6,708,058, “NORMAL CARDIAC RHYTHM TEMPLATE GENERATION SYSTEM AND METHOD,” assigned to Cardiac Pacemakers, Inc., which is hereby incorporated in its entirety. In one embodiment, the detected tachyarrhythmia is considered as “correlated” if a correlation coefficient exceeds a correlation threshold and as “marginally correlated” if the correlation coefficient exceeds a marginal correlation threshold that is lower than the correlation threshold. Correlation threshold adjuster 456 allows adjustment of the marginal correlation threshold. Tachyarrhythmia classifier 446 classifies the detected tachyarrhythmia using one or more of the atrial rate, ventricular rate, onset rate, stability parameter, and correlation coefficient. In one embodiment, tachyarrhythmia classifier 446 classifies the detected tachyarrhythmia using a method discussed below with reference to FIG. 5.

In one embodiment, tachyarrhythmia detector 444 performs a detection process that is initiated by a detection of three consecutive fast beats from the ventricular electrogram. In response to the detection of three consecutive fast beats, a tachyarrhythmia detection window is started. The tachyarrhythmia detection window includes ten consecutively detected heart beats starting with and including the three consecutive fast beats. If at least eight out of the ten heart beats in the tachyarrhythmia detection window are fast beats (i.e., the tachyarrhythmia detection window is satisfied), a tachyarrhythmia verification duration is started. Otherwise, the tachyarrhythmia verification duration is not started.

During the tachyarrhythmia verification duration, a moving verification window of ten consecutively detected heart beats is used to determine whether the detected tachyarrhythmia sustains. If at least six out of the ten heart beats in the verification window are fast beats (i.e., the verification window is satisfied), the detected tachyarrhythmia is considered to be sustaining. If this verification window fails to be satisfied at any time during the tachyarrhythmia verification duration, the tachyarrhythmia detection is terminated without delivering an anti-tachyarrhythmia therapy. If the detected tachyarrhythmia episode is determined to be sustaining throughout the tachyarrhythmia verification duration, it is classified by tachyarrhythmia classifier 446 to determine the necessity and type of an anti-tachyarrhythmia therapy. If the detected tachyarrhythmia is classified as a type of tachyarrhythmia for which a gene therapy is to be delivered, such as AF, the one or more gene regulatory signals are delivered while the AF is being detected. If the detected tachyarrhythmia is classified as a type of tachyarrhythmia for which a defibrillation therapy is to be delivered, such as VT or VF, a defibrillation therapy is delivered. Following the delivery of the defibrillation therapy, the tachyarrhythmia is redetected by repeating the detection and classification process or portions thereof.

FIG. 5 is a flow chart illustrating a method 500 for classifying a detected tachyarrhythmia. In one embodiment, tachyarrhythmia classifier 446 performs method 500. The atrial rate, ventricular rate, onset rate, stability parameter, correlation coefficient, and various thresholds used in method 500 are detected, produced, or programmed as discussed with reference to FIG. 4 above. For correlation analysis, the template waveform is produced using a cardiac signal sensed during an NSR.

A tachyarrhythmia is detected at 510, when the ventricular rate is within a predetermined tachyarrhythmia rate zone. If the ventricular rate (V-RATE) exceeds the atrial rate (A-RATE) by a predetermined margin at 512, the detected tachyarrhythmia is classified as VT. If the ventricular rate does not exceed the atrial rate by a predetermined margin at 512, and the correlation coefficient (FCC) exceeds the correlation threshold (FCC_{TH}) at 514, the detected tachyarrhythmia is classified as SVT. In one embodiment, the correlation threshold (FCC_{TH}) is programmable between 0.6 and 0.99, with approximately 0.94 being a specific example.

If the atrial rate does not exceed a predetermined threshold atrial rate (A-RATE_{TH}) at 516, the onset rate indicates a gradual onset of tachyarrhythmia at 518, and the correlation coefficient exceeds a first marginal correlation threshold (FCC_{MTH1}) (i.e., FCC falls between FCC_{MTH1} and FCC_{TH}) at 518, the detected tachyarrhythmia is classified as ST. ST is a physiologic tachyarrhythmia originated in an SA node when the SA node generates the electrical impulses at a tachyarrhythmic rate. In one embodiment, the first marginal correlation coefficient is programmable between 0.4 and the correlation threshold (i.e., 0.4≦FCC_{MTH1}≦FCC_{TH}), with approximately 0.8 being a specific example. In one embodiment, the first marginal correlation threshold is set to be lower than the correlation threshold by a predetermined amount, such as approximately 0.2 (i.e., FCC_{MTH1}≈FCC_{TH}−0.2).

If the correlation coefficient does not exceed the correlation threshold at 514, the atrial rate exceeds a predetermined threshold atrial rate at 516, and the ventricular rate is unstable at 522, the detected tachyarrhythmia is classified as AF. If the ventricular rate is stable at 522, the atrial rate exceeds the ventricular rate by a predetermined margin, and the correlation coefficient exceeds a second marginal correlation threshold (FCC_{MTH2}) (i.e., FCC falls between FCC_{MTH2} and FCC_{TH}) at 524, the detected tachyarrhythmia is classified as AFL. In one embodiment, the second marginal correlation threshold is programmable between 0.4 and the correlation threshold (i.e., 0.4≦FCC_{MTH2}≦FCC_{TH}), with approximately 0.8 being a specific example. In one embodiment, the second marginal correlation threshold is set to be lower than the correlation threshold by a predetermined amount, such as approximately 0.2 (i.e., FCC_{MTH2}=FCC_{TH}−0.2).

If the atrial rate approximately equals to the ventricular rate at 520, the onset rate indicates a sudden onset of tachyarrhythmia, the atrial and ventricular events occur in a specified SVT pattern, and the correlation coefficient exceeds a third marginal correlation threshold (FCC_{MTH3}) (i.e., FCC falls between FCC_{MTH3} and FCC_{TH}) at 520, the detected tachyarrhythmia is classified as AT. The detection of cardiac event patterns including the SVT pattern is discussed in U.S. patent application Ser. No. 11/276,213, entitled “RHYTHM DISCRIMINATION OF SUDDEN ONSET AND ONE-TO-ONE TACHYARRHYTHMIA”, filed on Feb. 17, 2006, assigned to Cardiac Pacemakers, Inc., which is hereby incorporated in its entirety. If these conditions are not met at 520, the detected tachyarrhythmia is classified as VT. AT is a pathologic tachyarrhythmia that occurs when a biologic pacemaker (focus) in an atrium usurps control of the heart rate from the SA node. In one embodiment, the third marginal correlation threshold (FCC_{MTH3}) is programmable between 0.4 and the correlation threshold (i.e., 0.4≦FCC_{MTH3}≦FCC_{TH}), with approximately 0.6 being a specific example. In one embodiment, the third marginal correlation threshold is set to be lower than the correlation threshold by a predetermined amount, such as approximately 0.4 (i.e., FCC_{MTH3}≈FCC_{TH}−0.2).

FIG. 6 is a block diagram illustrating an embodiment of a gene regulatory system 600, which represents a specific embodiment of system 300. Gene regulatory system 600 provides for control of the AV conduction during AF and includes implantable gene regulatory signal delivery device 330, a control circuit 632, and an AF detector 634. AF detector 634 detects AF episodes. In one embodiment, AF detector 634 represents portions of tachyarrhythmia detection and classification circuit 334 that detects AF episodes. Control circuit 632 includes command receiver 336. In response to the detection of AF by AF detector 634 and/or the external command received by command receiver 336, control circuit 632 initiates the delivery of the one or more gene regulatory signals from implantable gene regulatory signal delivery device 330. After the AF is no longer detected by AF detector 634, which indicates the end of the AF episode, control circuit 632 stops the delivery of the one or more gene regulatory signals. In one embodiment, control circuit 632 stops the delivery of the one or more gene regulatory signals after a predetermined time interval following the end of the AF episode as indicated by AF detector 634.

FIG. 7 is a block diagram illustrating an embodiment of a gene regulatory system 700, which represents a specific embodiment of system 600. Gene regulatory system 700 provides for control of AV conduction during AF and includes an LED 730, a control circuit 732, and AF detector 634. LED 730 represents one or more light emitting diodes (LEDs) that emit a light having characteristics suitable for controlling the AV conduction by the gene regulation discussed above. In one embodiment, the wavelength of the light is within the range of 350 to 750 nanometers, with 600 to 700 nanometers being a specific example. In one embodiment, the intensity of the light is controlled by controlling the voltage across LED 730 and/or the current flowing through LED 730. In one embodiment, LED 730 includes an array of LEDs that can be programmed to emit lights having one or more distinct wavelengths. Control circuit 732 includes command receiver 336. In response to the detection of AF by AF detector 634 and/or the external command received by command receiver 336, control circuit 732 initiates emission of the light from LED 730. After the AF is no longer detected by AF detector 634, which indicates the end of the AF episode, control circuit 732 stops the emission of the light. In one embodiment, control circuit 732 stops the emission of the light after a predetermined time interval following the end of the AF episode as detected by AF detector 634.

In one embodiment, LED 730 emits a light having a first wavelength to turn on a gene expression and a second wavelength to turn off the gene expression. In one embodiment, the first wavelength is within the range of 640 to 700 nanometers, with 670 nanometers being a specific example, and the second wavelength is within the range of 720 to 780 nanometers, with 750 nanometers being a specific example.

In one embodiment, control circuit 732 and AF detector 634 are housed in an implantable medical device such as implantable medical device 105 or 205. Control circuit 732 is electrically connected to LED 730 by conductors in a lead such as lead 225.

FIG. 8 is a block diagram illustrating an embodiment of a gene regulatory system 800, which represents another specific embodiment of system 600. Gene regulatory system 800 performs substantially identical functions as system 700 but has a different physical structure. Gene regulatory system 800 includes a light emission terminal 830, an optic fiber 836, a control circuit 832 including an LED 838, and AF detector 634. LED 838 represents one or more light emitting diodes having substantially the same electrical and optical characteristics as LED 730. In one embodiment, the intensity of the light is controlled by controlling the voltage across LED 838 and/or the current flowing through LED 838. In one embodiment, LED 838 includes an array of LEDs that can be programmed to emit lights having one or more distinct wavelengths. Control circuit 832 includes command receiver 336. In response to the detection of AF by AF detector 634 and/or the external command received by command receiver 336, control circuit 832 initiates emission of the light from LED 838. The light is transmitted through optic fiber 836 and delivered to the AV node from light emission terminal 830 at a distal end of optic fiber 836. After the AF is no longer detected by AF detector 634, which indicates the end of the AF episode, control circuit 832 stops the emission of the light. In one embodiment, control circuit 832 stops the emission of the light after a predetermined time interval following the end of the AF episode as detected by AF detector 634.

In one embodiment, control circuit 832 and AF detector 634 are housed in an implantable medical device such as implantable medical device 105 or 205. Light emission terminal 830 is incorporated into the distal end, or any other portion, of a lead such as lead 225 for placement over the AV node. Optic fiber 836 extends with the lead to provide for the optical connection between LED 838 and light emission terminal 830.

FIG. 9 is a block diagram illustrating an embodiment of a gene regulatory system 900, which represents another specific embodiment of system 600. Gene regulatory system 900 performs substantially identical functions as system 700 but has a different physical structure. Gene regulatory system 900 provides for control of AV conduction during AF and includes an implantable light emission device 940, a control circuit 932, and AF detector 634. Implantable light emission device 940 is wirelessly coupled to control circuit 732 via a telemetry link 944 and includes an LED 930 and a power source 942. LED 930 represents one or more light emitting diodes having substantially the same electrical and optical characteristics as LED 730. In one embodiment, the intensity of the light is controlled by controlling the voltage across LED 930 and/or the current flowing through LED 930. In one embodiment, LED 930 includes an array of LEDs that can be programmed to emit lights having one or more distinct wavelengths. Power source 942 supplies the energy required for the operation of implantable light emission device 940. In one embodiment, power source 942 includes a battery. In another embodiment, power source 942 includes a power receiver and converter to receive energy via telemetry link 944 or another power transmission link, such as an inductive or ultrasonic link, and converts the power to a form suitable for powering implantable light emission device 940. Control circuit 932 includes command receiver 336. In response to the detection of AF by AF detector 634 and/or the external command received by command receiver 336, control circuit 932 initiates emission of the light from LED 930. After the AF is no longer detected by AF detector 634, which indicates the end of the AF episode, control circuit 932 stops the emission of the light. In one embodiment, control circuit 932 stops the emission of the light after a predetermined time interval following the end of the AF episode as detected by AF detector 634.

In one embodiment, control circuit 932 and AF detector 634 are housed in an implantable medical device such as implantable medical device 105 or 205. Implantable light emission device 940 is placed over the AV node and wirelessly coupled to the implantable medical device.

FIG. 10 is a block diagram illustrating an embodiment of a gene regulatory system 1000, which represents another specific embodiment of system 600. System 1000 includes implantable gene regulatory signal delivery device 330, a control circuit 1032, tachyarrhythmia detection and classification circuit 334, and a pacing circuit 1038. Pacing circuit 1038 delivers pacing pulses through electrodes, such as those illustrated in FIG. 2. Control circuit 1032 is represents a specific embodiment of control circuit 332 and controls the delivery of the pacing pulses in addition to performing the functions of control circuit 332. In various embodiments, system 1000 provides for cardiac pacing therapies in addition to the gene regulatory therapies provided by any of systems 300, 600, 700, 800, and 900.

In one embodiment, while the AV conduction is slowed or blocked using implantable gene regulatory signal delivery device 330, ventricular pacing pulses are delivered from pacing circuit 1038, such as in a VVI pacing mode, to maintain a desirable ventricular rate.

While transient control of the AV conduction by the gene regulation discussed as a specific example, each of systems 100, 200, 300, 600, 700, 800, 900, and 1000 may be used to provide transient control of conduction in any of the electrical conductive pathways in the heart. In various embodiments, the one or more gene regulatory signals discussed in this document are applied to a target site on or near a cardiac electrical conduction pathway to temporarily slow or block conduction in that pathway.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed:

1. A system coupled to a heart having a right atrium (RA) and an atrioventricular (AV) node, the system comprising:
an implantable gene regulatory signal delivery device configured to deliver a light to a target site in the heart to transiently control an aberrant cardiac electrical conduction, the light having characteristics suitable for regulating a transcription control element; and
an implantable medical device communicatively coupled to the implantable gene regulatory signal delivery device, the implantable medical device including:
an atrial fibrillation (AF) detector configured to detect AF; and
a control circuit configured to initiate an emission of the light from the implantable gene regulatory signal delivery device in response to the detection of AF.

2. The system of claim 1, wherein the control circuit comprises a command receiver configured to receive an external command, and the control circuit is configured to initiate the emission of the light in response to one or more of the detection of AF and the reception of the external command.

3. The system of claim 1, comprising one or more light emitting diodes (LEDs) configured to emit the light.

4. The system of claim 3, wherein the one or more LEDs are configured to emit a light having a wavelength from 600 up to 700 nanometers.

5. The system of claim 3, comprising an implantable intracardiac lead having a proximal end, a distal end, and an elongate body coupled between the proximal end and the distal end, the distal end configured to be placed in the target site, and wherein the implantable gene regulatory signal delivery device is incorporated into the distal end of the implantable intracardiac lead, and the implantable medical device is coupled to the proximal end of the implantable intracardiac lead.

6. The system of claim 5, wherein the implantable gene regulatory signal delivery device comprises the one or more LEDs, and the implantable intracardiac lead comprises conductors extending within the elongate body and electrically connecting the one or more LEDs to the control circuit.

7. The system of claim 5, wherein the control circuit comprises the one or more LEDs, and the implantable intracardiac lead comprises at least one optic fiber extending within the elongate body and configured to transmit the light to the implantable gene regulatory signal delivery device to deliver to the target site.

8. The system of claim 3, wherein the implantable medical device is wirelessly coupled to the implantable gene regulatory signal delivery device via telemetry, and the implantable gene regulatory signal delivery device comprises a power source and the one or more LEDs.

9. The system of claim 5, wherein the implantable intracardiac lead is configured to allow placement of the distal end in the RA over the AV node, and the implantable gene regulatory signal delivery device is configured to deliver the light to the AV node to transiently control an aberrant AV conduction.

10. The system of claim 5, wherein the implantable intracardiac lead is configured to deliver light to the pulmonary veins.

11. A method to transiently control aberrant AV conduction, comprising:
delivering an amount of a regulatory signal to a mammal effective to transiently control aberrant AV conduction in cardiac cells having an expression cassette, wherein the expression cassette comprises a regulatable transcription control element operably linked to a nucleic acid sequence for a gene product which alters conduction, wherein the gene product comprises nucleic acid sequences corresponding to those for an inhibitor of beta-adrenergic receptor, an inhibitor of G_{S}, a Gᵢ protein, a dominant negative inhibitor of an ion channel, or a dominant negative inhibitor of gap junction, wherein the regulatory signal is a drug or energy from a device and wherein delivery of the regulatory signal increases expression from the regulatable transcription control element.

12. A method to transiently control cardiac arrhythmias, comprising:
administering to a mammal having or at risk of cardiac arrhythmias, an expression cassette comprising a regulatable transcription control element operably linked to a nucleic acid sequence for a gene product, wherein the gene product comprises nucleic acid sequences corresponding to those for an inhibitor of beta-adrenergic receptor, an inhibitor of G_{S}, a Gᵢ protein, a dominant negative inhibitor of an ion channel, or a dominant negative inhibitor of a gap junction, wherein the regulatable transcription control element is regulated by a regulatory signal, and wherein the regulatory signal is a drug or energy from a device; and
delivering to the mammal the regulatory signal in an amount effective to express the gene product, thereby transiently controlling cardiac arrhythmia in the mammal.

13. The method of claim 11 or 12 further comprising delivering the regulatory signal in response to detection of a physiological signal indicative of aberrant atrial rate, AV conduction or cardiac arrhythmia.

14. The method of claim 11 or 12 wherein the regulatable transcription control element comprises a regulatable promoter.

15. The method of claim 11 or 12 wherein the gene product is RNAi or an antisense oligonucleotide.

16. The method of claim 11 or 12 wherein the gene product encodes a Gi protein.

17. The method of claim 11 or 12 wherein the gene product is a dominant negative protein.

18. The method of claim 17 wherein the gene product encodes a dominant negative Cx40, Cx43, Cx45, HCN or G_{αi2}.

19. The method of claim 11 or 12 wherein the gene product is a calcium channel inhibiting G protein.

20. The method of claim 11 or 12 wherein the regulatable transcription control element is regulated by selected wavelengths of light.

21. The method of claim 11 or 12 wherein the regulatable transcription control element is regulated by a drug.

22. The method of claim 12 wherein the expression cassette is systemically administered to the mammal.

23. The method of claim 12 wherein the expression cassette is administered to an artery or coronary vein.

24. The method of claim 12 wherein the expression cassette is injected into selected areas of the atria of the mammal.

25. The method of claim 12 wherein a viral vector delivers the expression cassette to the mammal.

26. The method of claim 11 or 12 wherein the device is one or more implanted leads or a CRM device.

27. The method of claim 11 or 12 wherein the expression cassette is administered to the SA node or fat pads over the AV node of the mammal.

28. The method of claim 11 or 12 wherein aberrant conduction is blocked.

29. The method of claim 11 or 12 wherein aberrant conduction is inhibited.

30. The method of claim 11 or 12 wherein the regulatable transcription control element comprises a tissue-specific transcription control element.
